(19) United States
(12) Patent Application Publication
(10) Pub. No.: US 2017/0226501 A1
(43) Pub. Date: Aug. 10, 2017

(54) HYBRIDISATION COLUMN FOR NUCLEIC ACID ENRICHMENT

(71) Applicant: OXFORD GENE TECHNOLOGY (OPERATIONS) LTD., Begbroke, Oxfordshire (GB)

(72) Inventors: Oliver MILLER, Oxfordshire (GB); Graham SPEIGHT, Oxfordshire (GB); Ed SOUTHERN, Oxfordshire (GB); Mike MCANDREW, Oxfordshire (GB); Douglas HURD, Oxfordshire (GB)

(73) Assignee: OXFORD GENE TECHNOLOGY (OPERATIONS) LTD., Begbroke, Oxfordshire (GB)

(21) Appl. No.: 15/503,480

(22) PCT Filed: Aug. 14, 2015
(86) PCT No.: PCT/GB2015/052370

§ 371 Date: Feb. 13, 2017

(30) Foreign Application Priority Data
Aug. 14, 2014 (GB) 1414451.3

(51) Int. Cl. C12N 15/10; B01L 3/00; B01L 7/00

(52) U.S. Cl. CPC C12N 15/101; B01L 7/52; B01L 3/502707; B01L 2200/147; B01L 2200/0689; B01L 2300/0681; B01L 2400/0487; B01L 2300/0663

(57) ABSTRACT

The invention relates to the rapid enrichment of nucleic acid molecules of interest from complex mixtures of nucleic acids for the purpose of sequencing genes and variants, e.g. for clinical uses as well as other applications. A hybridisation column comprising an inner channel, wherein a portion of said channel is filled completely with a porous solid support comprising (a) a plurality of interconnected, micron-sized voids that permit a fluid to flow between them and the remainder of the channel, and (b) a plurality of hybridisation probes, which are bound to the surfaces of the solid support forming the voids is disclosed.

### FIELD OF THE INVENTION

The invention relates to the rapid enrichment of nucleic acid molecules of interest from complex mixtures of nucleic acids for the purpose of sequencing genes and variants, e.g. for clinical uses as well as other applications.

### BACKGROUND

There is a strong drive to implement genomic technologies into routine healthcare to improve diagnosis and drive stratified medicine (also known as personalised or precision medicine). The first step in any targeted sequencing assay is to enrich the sequences of interest from a mixture of whole genomic DNA or RNA. Paramount in diagnostics is the quality of data and robustness and speed of the assay. Current enrichment assays are not fit for clinical purposes as they are slow, or miss variants.

Commercially available amplicon (PCR)-based enrichment methods are prone to bias, can be unreliable in detecting variants, and are limited in the total numbers of regions that can be assayed. Enrichment methods based on hybridisation using a bait library offer better quality data and greater flexibility, but at the cost of time: in order to sequence specific genes in a sample of genomic DNA, the fragments that correspond to the regions containing the genes of interest must be extracted or enriched. Typically, in hybridisation-based enrichment methods, a set of biotinylated hybridisation probes (the bait library) are hybridised with the genomic DNA sample in solution. Subsequently, the bait-target duplexes are captured from solution using streptavidin-conjugated microbeads, while non-targeted DNA is not captured and is washed away. The targeted DNA is then amplified by PCR and sequenced. The entire workflow from DNA sample preparation to sequencing typically take numerous days to complete, compared to hours for amplicon-based methods.

The lengthiest step in hybridisation-based enrichment methods employing a bait library is the actual hybridisation step. The diffusion constant of DNA in water is extremely low. For example, the diffusion length of an 80 bp fragment in water is 1.9 mm in one day. In a microarray experiment where the height of the target solution above the probe array is hundreds of microns, an overnight incubation is typically needed to allow sufficient numbers of target molecules to diffuse close enough to the probes for hybridisation reactions to occur.

The object of the invention is to provide a process that can compete in terms of speed with amplicon-based methods, while avoiding the problems of poor reliability associated with these methods. In particular, the invention aims at providing a method for enabling hybridisation-based enrichment of nucleic acid samples for sequencing in less than a single day.

### SUMMARY OF THE INVENTION

The invention addresses the aforementioned deficiencies in the art by providing a hybridisation-based method for rapidly enriching specific sequences from samples of nucleic acids. The invention leverages microfluidics and surface hybridisation to accelerate the selective capture and processing of nucleic acid molecules from a mixture of nucleic acids. Using the process of the invention, a panel of DNA sequences from genomic DNA can be highly enriched allowing the resulting DNA to be sequenced quickly, cheaply, and with uniform coverage.

It is known in the prior art that, by reducing the height of the liquid layer covering the hybridisation probes, the rate of hybridisation is accelerated and microarray experiments can be completed in minutes, rather than days (see references 1, 2, 3). Reducing the diffusion length ensures more opportunities for target molecules to hybridise with the hybridisation probes. Narrow microfluidic channels have also been exploited to accelerate the detection of fluorescent oligonucleotides when the channels existed as voids between microbeads in a packed bed (see reference 4). In this case, probes were conjugated to the microbeads and the target solution was passed between them, permitting hybridisation times on the order of a few minutes.

To date, narrow, micron-scale microfluidic channels have not been exploited to accelerate the solid-phase capture of specific DNA fragments from a sample for subsequent sequencing. In reference 4, constrained diffusion lengths were used to accelerate the detection of specific sequences, not accelerate their enrichment for other purposes, such as sequencing. Microarrays have been employed for enriching DNA samples prior to sequencing (see references 5, 6 and 7), but a solid support with a multitude of micron-scale voids to drive hybridisation were not used and so overnight hybridisations were necessary.

Reference 8 attempted to address the deficiencies of the prior art method by providing a micro spin column that employs hybridisation probes coupled to a glass microfiber filter to enrich fragmented genomic DNA for exonic sequences of interest. However, despite the suggested advantages of high sequence enrichment efficiency, enrichment capacity and high specificity, reference 8 falls short of providing a hybridisation-based enrichment method that requires hybridisation times in the order of a few minutes, rather than many hours.

The inventors discovered that rapid enrichment of a sample of fragmented genomic DNA can be achieved by driving the sample through a packed bed of microbeads which had coupled to their surfaces a mixture of oligonucleotide probes. The high surface area of the microbeads and the narrow, micron-scale voids between them ensure that the DNA fragments remain in close contact with the probes as they flow, permitting rapid hybridisation. Non-targeted DNA interacts only weakly with the probes and is flushed out of the microbead bed by the continuous flow of hybridisation buffer and, subsequently, a stringent wash buffer. After washing, the targeted DNA is released from the microbeads by raising the temperature or otherwise modifying the physicochemical environment. This eluted DNA is enriched for the targeted regions relative to the rest of the genome. The inventors found that, using this approach, the time required for enriching the targeted molecule in a sample of genomic DNA can be reduced to less than 2 hours.

The invention relates to a hybridisation column comprising an inner channel, wherein a portion of said channel is filled with a porous solid support. Typically, one end of the channel comprises a porous filter, frit or permeable membrane to keep the solid support in the column when suction is applied to said end of the channel or pressure is applied to the opposite end of the channel. The solid support fills the entire cross-section of the channel. The solid support comprises (a) a plurality of interconnected, micron-sized voids that permit a fluid to flow between them and the remainder of the channel, and (b) a plurality of hybridisation probes, which are bound to the surfaces of the solid support forming the voids. Preferably, the pore structure of the solid support is homogeneous in all dimensions. The voids within the solid support typically have an average pore size of 0.1-100 μm.

The solid support may have a volume that ranges from 0.1 mm³ to 100 mm³. In some embodiments, a substantial portion of the channel is occupied by the solid support. For example, ≧10%, preferably ≧30%, more preferably ≧60% of the channel may be occupied by the solid support. The voids within the solid support may take up about 30-50% of the volume taken up by the solid support in the channel.

The hybridisation column can be used to enrich nucleic acid molecules of interest from a mixture of nucleic acids. The nucleic acid molecules of interest bind specifically to the hybridisation probes, whereas the remaining nucleic acids in the fluid are washed away by the flow of fluid through the channel during use of the column.

The invention also relates to a microfluidic device comprising one or more of these hybridisation columns, a temperature control element and a temperature sensor, wherein the temperature control element can be used to control the temperature within the channel of the one or more hybridisation column(s).

The microfluidic device may also comprise one or more reservoir(s) each of which is connected to one end of the channel of the one or more hybridisation column(s). The reservoir can be sealed and pressurised, e.g. by supplying a source of compressed gas, which is connected to the reservoir via tubing. When pressure is applied to the sealed reservoir during the operation of the device, fluid that has been applied to the reservoir is pushed into the channel of the hybridisation column and through the solid support located within the hybridisation column. Advantageously, the microfluidic device also comprises a valve within the tubing which connects the source of the compressed gas to the reservoir. By opening and closing the valve, e.g. by means of a programmable electronic controller, the flow rate of fluid driven through the channel can be adjusted.

Alternatively, one end of the channel of the one or more hybridisation columns is connected to a suction pump, wherein the suction pump is connected to a controller, which can control the flow rate of fluid driven through the channel of the one or more hybridisation column(s) when suction is applied. For example, the device may include an electronic controller, which can be programmed to control the flow rate through the channel of the hybridisation column.

Typically, the microfluidic device also includes collection tubing at the other end of the channel, i.e. downstream of the solid support during use. The collection tubing can be attached to a collection vessel which collects any fluid that has passed through the solid support.

The invention also refers to a method of preparing the hybridisation columns described above. The method comprises providing a column comprising an inner channel, and filling the entire cross-section of the channel with a plurality of microbeads having about the same diameter and having linked to their surfaces a plurality of hybridisation probes, wherein the microbeads form a porous solid support. A kit for preparing the hybridisation columns of the invention may be provided which contains columns comprising an inner channel and a container comprising a plurality of microbeads having about the same diameter and having linked to their surfaces a plurality of hybridisation probes.

In another aspect, the invention relates to a method for enriching nucleic acid molecules from a complex mixture of nucleic acids. In a first step, the method comprises providing a solid support which comprises (a) a plurality of interconnected, micron-sized voids that permit a fluid to flow between them, and (b) a plurality of hybridisation probes, which are bound to the surfaces of the solid support forming the voids. In a second step, the mixture of nucleic acids is driven through the solid support thereby allowing nucleic acid molecules comprising nucleic acid sequences complementary to the nucleic acid sequences of the hybridisation probes to hybridise to the probes. While the sample is driven through the solid support, hybridisation is allowed to proceed. Typically, hybridisation times of less than 10 hours, preferably less than 5 hours, more preferably less than 1 hour are required. Preferably hybridisation time ranges from between 1 second and 1 hour, for example about 30 minutes or less, more preferably about 5 minutes or less, e.g. 30 seconds to 2 minutes. The mixture is typically driven through the solid support at a flow rate of 1-100 μl/minute. For hybridisation, the flow rate is adjusted based on the volume of the solid support to achieve 1-10 volume changes per minute, whereas a higher number of volume changes per minute is desirable for washing steps. Once the sample has been applied, a wash buffer is flushed through the solid support to remove any nucleic acids that are not hybridised to the probes. In a final step, the enriched nucleic acid molecules are recovered by eluting the nucleic acid molecules bound to the hybridisation probes from the solid support.

The steps of hybridisation, washing and elution can be performed at the same temperature, but typically enrichment is much more efficient if the temperature is optimised for each step. For example, hybridisation usually takes place at a temperature of 55-65° C. The washing step may be performed at a temperature 5-10° C. below the temperature used for hybridisation or at room temperature/ambient temperature (18-26° C.). Elution of the hybridised nucleic acid molecules can be achieved by heating the solid support to a temperature of about 90-100° C. Alternatively, hybridisation duplexes can be disrupted by physiochemical means such as modification of the pH.

In a further aspect of the invention, a method of preparing a sample comprising a mixture of nucleic acid molecules for high-throughput sequencing is provided. The method comprises contacting the sample with a solid support comprising one or more hybridisation probes under conditions that allow for binding of complementary nucleic acid sequences of the nucleic acid molecules and the hybridisation probes; removing unbound nucleic acid molecules; contacting the bound nucleic acid molecules with adapter molecules; joining the bound nucleic acid molecules to the adapter molecules to obtain adapter-terminated nucleic acid molecules; and releasing the adapter-terminated nucleic acid molecules from the solid support.

In another aspect of the invention, a method of preparing a sample comprising a mixture of nucleic acid molecules for high-throughput sequencing is provided. The method comprises contacting the sample with a solid support comprising one or more hybridisation probes under conditions that allow for binding of complementary nucleic acid sequences of the nucleic acid molecules and the hybridisation probes; removing unbound nucleic acid molecules; releasing the bound nucleic acid molecules from the solid support; contacting the released nucleic acid molecules with adapter molecules; and joining the nucleic acid molecules to the adapter molecules to obtain adapter-terminated nucleic acid molecules.

In a further aspect, the invention relates to a microbead comprising more than one set of hybridisation probes, wherein each hybridisation probe in the same set binds to the same region of a genome of an organism, and each set binds to a different region in the genome. The different regions may be located in different parts of the genome (e.g. different coding or non-coding regions or on different chromosomes). The different regions can be in different genes, or they can be located within the same gene. In certain embodiments, the different regions may overlap with each other. The different regions typically are located within coding regions of the genome, and the coding regions may be associated with a disease or disorder. Examples of diseases or disorders include neoplastic diseases, neurological diseases, autoimmune diseases, metabolic diseases or disorders, genetic diseases and constitutional disorders. Alternatively, the different regions may be associated with one or more prenatal diseases or disorders, or hereditary or genetic disorders for which prenatal diagnosis is desired, e.g. to allow early detection and intervention. In some embodiments, a library comprising a plurality of these microbeads is provided.

In a further aspect, the invention provides a microbead comprising one (preferably more than one) set of hybridisation probes, wherein each hybridisation probe in a set binds to the same region of a genome and the region of complementarity of a hybridisation probe in a set overlaps with the region of complementarity of at least one other hybridisation probe within the same set, wherein each set represent a different region of the genome. Preferably, the overlap between each hybridisation probe provides a minimum tiling depth of 2×. The microbead may comprise at least 5, preferably at least 10, more preferably at least 20 different sets of hybridisation probes.

In some aspects, the invention relates to a library comprising a first microbead and a second microbead, wherein said first microbead is linked to a plurality of hybridisation probes which bind to a first region of complementarity in a genome of an organism, and wherein said second microbead binds to a second region of complementarity in the genome, wherein said first region and said second region are located in different regions of said genome. The first region and the second region may be located in different parts of the genome (e.g. different coding or non-coding regions or on different chromosomes). The different regions can be in different genes. The hybridisation probes which bind to the first region of complementarity and the hybridisation probes which bind to the second region of complementarity may all have the same sequence, or they may all have different sequences. The library may comprise more than two microbeads binding to more than two different regions of a genome. For example, the library may comprise at least 10 different microbeads that bind to 10 different regions of said genome, preferably at least 20, more preferably at least 50.

The invention also relates to a library comprising a first microbead and a second microbead, wherein said first microbead is linked to a first plurality of hybridisation probes which bind to a first oncogene in a genome of an organism, and wherein said second microbead is linked to a second plurality of hybridisation probes which bind to a second oncogene in the genome.

The invention further provides a library comprising more than 100 microbeads, wherein each microbead comprises a hybridisation probe that binds to a different region in a genome of an organism.

### DETAILED DESCRIPTION OF THE INVENTION

The invention relates to a process of enriching a sample for nucleic acid molecules of interest using hybridisation. The process combines aspects of the work described in reference 4 with hybridisation-based methods for enriching DNA prior to sequencing. The key differences to reference 4 are:
1. The nucleic acid molecules of interest are enriched for downstream applications such as amplification and/or sequencing rather than detection and therefore do not contain a fluorochrome or similar exogenous detectable label.
2. The solid support comprising the hybridisation probes is packed in a millimetre-scale channel in the microfluidic device of the invention and therefore provides a far greater surface area for nucleic acid molecules of interest to bind to hybridisation probes than the microbeads in the micron-scale chamber of reference 4.
3. The microfluidic device allows for the precise control of hybridisation conditions by providing means for controlling (i) the temperature in the channel comprising the solid support and (ii) the flow rate of the fluid that passes through the solid support, and thus the hybridisation time.
4. After hybridisation and washing are complete, the enriched nucleic acid molecules are released from the solid support, e.g. by an increase in temperature, and collected for downstream applications such as sequencing.
5. Specificity of capture is achieved with the microfluidic device of the invention through precise control of the hybridisation conditions within the solid support. Reference 4 does not envisage or allow for this. The hybridisation conditions provided by the microfluidic device disclosed in reference 4 are not suitable for the specific capture of nucleic acids from a complex pool where both specific and non-specific hybridisation occurs.

The microfluidic device of the invention differs from the micro spin column in reference 8 in that the glass microfiber filter disk which serves as the solid support for the attachment of hybridisation probes is prepared by cutting out a disk from a bigger filter using a paper punch. For the disk to be placed easily into the micro spin column, the filter disk must have a diameter that is slightly smaller than the inner bore of the micro spin column and therefore does not completely fill the entire cross-section. Part of a sample applied to the micro column therefore will pass through the column without entering into the filter disk. To allow for efficient hybridisation, the tube containing the column with the filter disk is placed into a rotator and incubated for 16 to 20 hours. In contrast, the hybridisation column of the invention makes it possible to apply a sample to a solid support without extended incubation times for hybridisation to occur because the entire sample is brought into direct contact with the solid support which fills the entire cross-section of the channel within the hybridisation column.

The differences to microarray-based enrichment methods are:
1. The solid support of the invention provides a far greater surface area for the attachment of hybridisation probes than a typical glass slide used for microarrays;
2. A network of interconnected microfluidic channels exists as micron-scale voids within the solid support preventing the hybridisation probes of interest from diffusing very far from the hybridisation probes, whereas the microarrays of the prior art comprise either as a single deep channel or a space above the surface containing the hybridisation probes; and
3. The hybridisation probes against the targeted nucleic acid molecules of interest are mixed together and attached to the surface of the solid support randomly, rather than as an ordered array of individual probe spots.

### Sample Preparation

The sample comprising the nucleic acid molecules of interest can be derived from various sources and typically is pre-processed to remove most other components that are not nucleic acid molecules of interest. The nucleic acid molecules in the sample can be either DNA or RNA. For example, if the nucleic acid molecule of interest is genomic DNA, then RNA molecules are removed prior to any further processing steps. Similarly, where the nucleic acid molecule of interest is RNA (e.g. for transcriptome profiling), any contaminating cellular DNA will be removed as well as any RNases that could degrade the RNA molecules of interest. In addition, RNA molecules may be reverse-transcribed into DNA before enrichment or they may be enriched directly without prior reverse transcription. In some embodiments, the nucleic acid molecule of interests may be those that are bound e.g. by a certain transcription factor of interest, in which case pre-processing may involve chromatin immunoprecipitation (ChIP) to isolate DNA fragments bound to the transcription factors from other parts of the genome.

In some embodiment, the nucleic acid molecules in the sample are modified prior to the enrichment process. For example, methylated DNA may be bisulphite-treated. This treatment converts cytosine residues to uracil, but leaves 5-methylcytosine residues unaffected.

The nucleic acid molecules can be modified by joining them to locked nucleic acids (LNAs), in which the ribose moiety contains an extra bridge connecting the 2′ oxygen and 4′ carbon. The locked ribose conformation enhances base stacking and backbone pre-organization and can improve target specificity and stability of modified nucleic acid molecules.

In some instances, it may also be advantageous to modify the nucleic acid molecules by joining them to peptide nucleic acids (PNAs). PNAs may serve as tags to increase the capture efficiency of the nucleic acid molecules.

Depending on the application, larger nucleic acid molecules may need to be fragmented into more manageable pieces to be suitable for enrichment by hybridisation and downstream applications such as amplification and sequencing. To be suitable for the enrichment process of the invention, the nucleic acids typically have a length of 10-1,000 bases. Nucleic acid molecules of 50-500 base pairs in length are particularly useful. In a preferred embodiment, the nucleic acid molecules have a length of 100-350 bases (e.g. 150-200 bases). Shorter sequences (<1,000 bases, preferably <500 bases, more preferably <300 bases) hybridise more efficiently. Sequences that are too short (<50 bases, e.g. <25 bases or <10 bases) may make downstream processes such as sequencing inefficient.

In general, the limit on target length is dependent on factors such as binding capacity of the hybridisation probes on the void surfaces, etc. The size of the nucleic acid molecules may also depend on the desired application. For example, the nucleic acid size of 100-350 bases may be useful to analyse single nucleotide polymorphisms (SNPs), single nucleotide variations (SNVs), and short insertions or deletions (indels), where the sequences of interest typically include the 50 bases surrounding the SNP, SNV or indel.

Large nucleic acid molecules can be fragmented by mechanical shearing, e.g. by sonication. In some embodiments, mechanically sheared, fragmented nucleic acid molecules are end-repaired and ligated to adapter sequences prior to enrichment by hybridisation. The adapter molecules contain the priming sites required for downstream applications such as amplification and sequencing. Adapter ligation and suitable adapter molecules are described below.

Genomic DNA can be randomly fragmented into double-stranded fragments, e.g. by ultrasonic shearing or nebulisation (see references 9 and 10). Typically, the genomic DNA is fragmented into pieces of <1000 base pairs (bp) in length. A suitable length is in the range of 50-500 bp, e.g. 100-350 bp. Fragments having a length of 150-200 bp are particularly suitable. These fragments need to be ligated to adapters before they can be used for amplification and sequencing. Alternatively, enzymatic methods may be used in place of mechanical shearing and ligation (e.g. Illumina's Nextera system, New England Biolabs' DNase I fragmentase). Transposase enzymes pre-loaded with the adapter sequences are added to large pieces of DNA. The transposase enzymes randomly cleave the large pieces into much shorter fragments, while simultaneously tagging them with the adapters. The adapters introduce a forward primer site and a reverse primer site. The fragments can then be cleaned before enriching them for DNA molecules of interest to remove any un-ligated adapter proteins and any ligase/transposase that remains in the sample. An optional step before enrichment might be to fill-in the overhangs in the tagged adapters. The resulting fragments can be enriched in the same way as pre-enrichment ligated DNA fragments.

Only single-stranded nucleic acid molecules are able to hybridise to the hybridisation probes used for enrichment. Double-stranded nucleic acid molecules need to be denatured prior to enrichment by hybridisation. The denaturation step is typically performed at 90-100° C., preferably at about 95° C., more preferably at about 98° C. A typical denaturation protocol for a DNA sample requires that the sample is incubated at 98° C. for 5 minutes, cooled to 65° C. for 5 minutes, and then stored at 4° C. for 5 minutes before the sample is applied to the solid support.

### Enrichment by Hybridisation

The enrichment process of the invention is based on the principle that, under stringent conditions, a first nucleic acid molecule will bind with high specificity to a second nucleic acid molecule, where the second nucleic acid molecule has a sequence that is complementary to the sequence of the first nucleic acid molecule. To enrich nucleic acid sequences of interest, a sample comprising a plurality of nucleic acid molecules is brought into close contact with hybridisation probes, which have nucleic acid sequences that are complementary to the nucleic acid sequences of interest. Under stringent conditions, a nucleic acid molecule comprising a nucleic acid sequence of interest will bind to a hybridisation probe that has a sequence that is complementary to the nucleic acid sequence of interest. The hybridisation probe is linked to a solid support. After addition of the sample, a wash buffer is applied to the solid support, washing away all nucleic acid molecules that are not bound to a hybridisation probe. Nucleic acid molecules containing a sequence of interest stay bound to the solid support via the hybridisation probes and can be eluted from the solid support, e.g. by increasing the temperature or the salt concentration of the washing buffer. The eluate is highly enriched for nucleic acid molecules containing sequences of interest.

### Hybridisation Column

The invention relates to a hybridisation column comprising an inner channel, wherein a portion of said channel is filled with a porous solid support. The total volume occupied by the solid support within the column can be up to 1.5 cm³, however more commonly, the volume ranges from 0.1 mm³ to 100 mm³, more preferably from 0.25 to 10 mm³. Typical volumes are 0.5 to 1.5 mm³. Typically, about 30-50% of the volume taken up by the solid support is void space because of the porous nature of the solid support of the invention. For example, if the volume taken up by the solid support is 1 mm³, the void space within the solid support is about 0.3-0.5 mm³.

The solid support completely fills the entire cross-section of the inner channel of the hybridisation column. The solid support may extend throughout a substantial portion along the depth of the channel, or may fill only a small portion of the channel's depth. Typically, the solid support has a depth of about 0.1-50 mm, preferably about 0.3-20 mm, whereby the depth refers to the dimension the solid support extends to within the channel relative to the direction of flow through the channel. The internal bore of the channel can be 100 μm to 20 mm, preferably 200 μm to 10 mm, more preferably 300 μm to 2 mm. The skilled person will appreciate that the length of the channel and its diameter will be chosen so as to accommodate a solid support of the desired volume.

The shape of the inner channel of the column may vary. For example, the inner bore of the channel may be wider on one end than on the other end, and may be tapered. Typically, the diameter of the inner channel will be the same throughout the hybridisation column. A circular cross-section is typical.

In some embodiments, the channel is tapered, and the tapered end is closed off by a porous filter or frit or a permeable membrane (as discussed above). For example, the channel may narrow from an internal bore of 5-10 mm to an internal bore of 50 μm to 1 mm. Narrowing the channel is advantageous because it allows the formation of a solid support with greater depth and hence nucleic acid molecules which pass through the solid support may interact with a larger number of hybridisation probes increasing the chances of a successful capture event. The larger headspace preceding the tapered part of the channel makes pressure control easier.

A hybridisation column in accordance with the invention can have various shapes and sizes. For example, a hybridisation column can be a thin and elongated capillary which includes the solid support of the invention. Alternatively, the hybridisation column can have a format similar to a conventional spin column or any other type of column which is conventionally used for chromatographic separation and purification. A typical hybridisation column (1) for use with the invention is shown in FIG. 7 and comprises an inner channel (2) and a porous frit (3), wherein said inner channel (2) is stepwise tapered, dividing the column into a top section (4), a neck section (5) and a bottom section (6), wherein the frit (3) is located in the bottom section (6) of the column (1) and obstructs the entire cross-section of the inner channel (2). The inner channel (2) may taper in the neck section (5) to about 30% to about 50% of the diameter of the inner channel (2) at the top section (4) of the column (1). The inner channel (2) may taper further in the bottom section (6) to about 20% to about 30% of the diameter of the inner channel (2) at the top section (4) of the column (1). In some embodiments, the top section (4) is divided into an upper top section and a lower top section by a rim (8) protruding into the inner channel (2). In certain embodiments, the bottom section (6) has a volume of about 10 μl to about 30 μl (e.g. about 20 μl). The top section (4) can make up about 60-75% of the total length of the column (1). The neck section (5) can make up about 5-10% of the total length of the column (1). The bottom section (6) can make up about 20-30% of the total length of the column (1).

Typically, the column is prepared from a material with low DNA binding capacity. qPCR with random primers can be used to assess whether a column has DNA bound to it. It is desirable that the column material conducts heat. Ideally, the hybridisation column is autoclavable to render it DNase- and RNase-free prior to use. Suitable column materials include low-DNA binding plastics made from polypropylene or polyallomers. Columns made from polypropylene or polyallomers also have thermal conductivity properties that render them suitable for practising the invention. Polyethylene-based columns may also be suitable for use with the invention. Preferably, the hybridisation column is not made from (stainless) steel or similar metals

In order to better control the conditions within the solid support during hybridisation, washing and elution, the hybridisation columns of the invention are preferably used with a microfluidic device according to the invention. However, in some aspects of the invention, the hybridisation columns can also be used to practice the methods of the invention without a dedicated microfluidic device.

For example, a sample containing nucleic acid molecules of interest may be applied to a hybridisation column manually, e.g. by pipetting the sample into the inner channel onto the solid support of the hybridisation column. To control the hybridisation conditions, the hybridisation buffer containing the sample can be pre-warmed and the hybridisation column can be placed into a hybridisation oven, heating block or the like to allow the hybridisation step to complete. Once the nucleic acid molecules of interest have been hybridised to the hybridisation probes on the solid support, the hybridisation buffer can be removed by placing the hybridisation column into a tube and applying a centrifugal force, so that any fluid remaining in the solid support is forced into the tube. Subsequent washing steps can be performed in the same way, i.e. by applying wash buffer to the solid support and removing the wash buffer by applying a centrifugal force. In each instance, the flow through in the tube will be discarded. Once washing has been completed, the hybridisation column can be placed in a fresh tube and elution buffer can be applied to the solid support. The enriched sample containing the nucleic acid molecules of interest can be eluted by placing the hybridisation column within the tube into a centrifuge and applying a centrifugal force. The enriched sample will be forced into the tube which then contains the enriched sample. In place of a centrifuge, the sample and the wash and elution buffers can also be forced through the column in the solid support by applying pressure or suction to the inner channel of the hybridisation column. For example, the hybridisation column may be fitted with a Luer lock to allow a syringe to be connected to it, which can be used to drive the sample and buffers through the solid support.

### Solid Support

The solid support of the invention is porous to provide a high surface area to which the hybridisation probes are bound. The term “porous” in this context refers to the micron-sized voids within the solid support, which are interconnected to permit a fluid to flow between them, rather than any pores within the material from which the solid support is formed. The solid support can be formed from a material that in itself is either porous or non-porous in nature. For example, the solid support may be formed from glass microspheres which may be porous themselves. Preferably, however, the solid support is formed from non-porous materials such as metallic or silica microbeads because the presence of pores within the material forming the solid support may trap nucleic acids applied to the solid support, slow down the enrichment process and reduce recovery of enriched nucleic acids from the solid support. A typical solid support in accordance with the invention provides a surface area of about 600 to 800 mm² per mm³ (corresponding to a surface area-to-volume ratio of 600 to 800).

Once a sample containing nucleic acid molecule of interest is applied to the solid support, the micron-scale voids within it ensure that the nucleic acid molecules come into close contact with the hybridisation probes as they flow through the solid support, enabling rapid hybridisation. Non-targeted nucleic acid molecules interact only weakly with the hybridisation probes and are flushed out of the solid support by the continuous flow of hybridisation buffer and, subsequently, a stringent wash buffer.

The voids within the solid support may have irregular shapes and their size may vary slightly throughout the solid support. For porous materials, average pore size can be used to define the characteristics of the voids within the solid support. A practical approach for determining the notional “average pore size” of a solid support according to the invention is the step-by-step addition of particles of increasing size to the flow through the channel of the microfluidic device that contains the solid support. It will be appreciated that the particles used for testing will be defined by their average diameter. To determine the average pore size, all particles in the test population will have about the same diameter. In this context, a population of particles with about the same diameter refers to a population of particles of which 85% by number have a diameter within ±10% of the median diameter by number.

Once a particle size is reached where less than 10% of the particles that were added to the flow are recovered in the fluid exiting the solid support, this particle size can be used to define the average pore size of the solid support. For example, a solid support that does not retain microparticles having an average size of 9 μm, but retains more than 90% of particles having an average size of 10 μm can be defined as having an average pore size of 10 μm. The voids in the solid support of the invention can have an average pore size of e.g. 50 μm, 30 μm or 10 μm. Typical average pore sizes fall within the range of 0.1-100 μm. In some embodiments, the voids within the solid support of the invention have an average pore size of ≦2 μm, preferably ≦1.5 μm, more preferably ≦1 μm.

Preferably, the pore structure of the solid support is homogeneous in all dimensions. For example, a bed of tightly packed microbeads or a ceramic filter will have the same range of pore sizes at any cross-section, whereas a solid support made from microfibers will have a different range of pore sizes depending on whether the cross-section is perpendicular or parallel to the direction in which the microfibers are laid out within the solid support. Having the same range of pore sizes throughout the solid support provides consistent binding conditions at any point within the solid support as a nucleic acid molecule moves through it. Because of the small diffusion distances within the micron-sized voids, hybridisation of a nucleic acid molecule of interest is virtually guaranteed as it almost certainly will encounter a complementary hybridisation probe on its path through the solid support.

The voids within the solid support form interconnected, microfluidic paths. To increase the likelihood that every nucleic acid molecule within the sample is brought into contact with a hybridisation probe, the hybridisation probes are randomly distributed on the surface of the solid support. Preferably the hybridisation probes for a particular nucleic acid molecule of interest are not in a spatially defined location within the solid support, but can be found at substantially equal density throughout the solid support.

To achieve a high surface area to which hybridisation probes can be bound, the solid support may be formed from microfibers (i.e. a synthetic fibre finer than one or 1.3 denier or decitex/thread), porous glass microspheres (which typically have a diameter of 1 μm to 1 mm), microbeads (which typically have a diameter of 0.5 to 500 μm), ceramic filters etc. but beads or spheres are preferred. The solid support can be made of various substrate including glass, quartz, mica, carbon, apatite, alumina, silica, silicon carbide, silicon nitride, boron carbide, graphite, polycarbonate, polypropylene, polyamide, phenol resin, epoxy resin, polycarbodiimide resin, polyvinyl chloride, polyvinylidene fluoride, polyethylene fluoride, polyimide, acrylate resin etc. The substrate surface on the solid support may need to be functionalised to allow for the attachment of the hybridisation probes, either directly or via a linker group, using methods well-known in the art.

Typically, the solid support is provided as part of a hybridisation column wherein the column comprises an inner channel the entire cross-section of which is filled with the solid support. In some cases, the material for preparing the solid support (e.g. microbeads to which hybridisation probes have been attached) is provided separately from the column and the hybridisation column comprising the solid support will be prepared by the end user. In one aspect of the invention, a method of preparing a hybridisation column comprising the solid support of the invention is provided. The method comprises providing a column which comprises an inner channel and filling the entire cross-section of the channel with a plurality of microbeads having about the same diameter, so that the microbeads form a porous solid support.

### Microbeads

In a preferred embodiment, the solid support is formed by microbeads. In some embodiments, the microbeads are magnetic. In other embodiments, the microbeads are non-magnetic. Using microbeads provides several advantages. Microbeads are readily available commercially and come functionalised with various groups and in various sizes. Hybridisation probes can be attached to microbeads using methods known in the art. Typically, the hybridisation probes are attached covalently to the functionalised microbeads. For example, nucleic acid molecules having a 5′ amine modification can be readily linked to NHS-functionalised microbeads. As an alternative to covalently attaching nucleic acid molecules to microbeads, a high-affinity, non-covalent attachment mean can be used to link the nucleic acid to the microbeads. For example, streptavidin-functionalised microbeads can be conjugated to nucleic acid molecules that are linked to biotin.

Microbeads can be tightly packed to form a porous solid support with micron-scale voids. The size of the voids is determined by the size of the microbeads used, and hence depending on the application, the size can easily be varied, e.g. by employing larger microbeads to create bigger voids. For example, applications employing larger nucleic acid fragments (>500 bases) such as fragmented genomic DNA may require larger voids than applications using smaller fragment sizes (<300 bases). Thus, a hybridisation column containing the solid support can easily be adapted for use with larger fragments by choosing a larger microbead of the same material.

Typically microbeads have an average diameter of 0.5 to 500 μm. Microbeads having an average diameter of ≦20 μm are particularly useful. For the use with the present invention, the microbeads are preferably 2-10 μm in average diameter, more preferably, 4-8 μm. Suitable microbeads have an average diameter of approximately 5 μm. Where an average diameter is given, all microbeads in a solid support will have about the same diameter. For a plurality of microbeads with about the same average diameter, 85% by number of microbeads have a diameter within ±10% of the median diameter by number. By way of example, Dynabeads (Life Technologies, Inc) M-280 streptavidin microbeads have a diameter of 2.8 μm and CV<3%.

A further advantage of working with microbeads is that they can be linked to tens, hundreds, or thousands of different hybridisation probes without hindering hybridisation reactions with neighbouring probes. Linking a mixture of different hybridisation probes to functionalised microbeads results in the random distribution of each individual hybridisation probe in the mixture on the surface of the bead. As the solid support is formed of hundreds or thousands of microbeads, uniform distribution of individual microbead probes throughout the solid support is achieved.

A microbead may comprise more than one set of hybridisation probes. Each hybridisation probe in the same set can bind to the same region of a genome of an organism, and each set is designed to bind to a different region in the genome. The different regions may be located in distinct part of the genome (e.g. in different coding or non-coding regions or genes, or on different chromosomes). The hybridisation probes in a set bind to the same region of the genome, but they may not have all the same sequences. For examples, the hybridisation probes may be tiled across the region, and each hybridisation probe may overlap with neighbouring probes, e.g. by 10 or more nucleic acids, to cover the entire regions to a tiling depth of at least 2×, preferably at least 4×, more preferably at least 8× (e.g. 10× or more). As an alternative, hybridisation probes may be spaced 10-50 bases or even 100 bases or more than 1000 bases apart. The spacing between probes may depend on the downstream application. For example, nanopore sequencing achieves reads of up to 10 kilobases in length, in which case hybridisation probes may be spaced about 1000 bases to about 10,000 bases apart, e.g. about 2000-8000 bases apart or 3000-5000 bases apart.

The hybridisation probes may be designed to bind to regions of complementarity within a genome of interest, in particular in coding regions of the genome that may be associated with a disease or disorder. Examples of diseases or disorders include neoplastic diseases, neurological diseases, autoimmune diseases, metabolic diseases or disorders, genetic diseases and constitutional disorders. Alternatively, the different regions may be associated with one or more prenatal diseases or disorders, or hereditary or genetic disorders for which prenatal diagnosis is desired, e.g. to allow for early detection and intervention. Detection of aneuploidy, in particular trisomy 13, trisomy 18 and trisomy 21, and Turner's syndrome is particularly useful. In some embodiments, the microbeads of the invention may be used in cancer diagnostics to detect mutations in oncogenes. For example, the hybridisation probes may bind to mutated version of genes associated with tumour progression that can be detected in circulating tumour DNA. A microbead may comprise at least 5, preferably at least 10, more preferably at least 20 different sets of hybridisation probes. In some embodiments, a microbead may comprise 100, preferably 500, more preferably 1000 different sets of hybridisation probes.

Alternatively, microbeads can be linked to tens, hundreds, or thousands of the same hybridisation probe and the solid support can be formed by mixing microbeads linked to individual hybridisation probes. In some embodiments, the hybridisation probes are not all the same, but all bind to the same region of a genome. For example, each hybridisation probe may overlap with neighbouring probes, e.g. by 10 or more nucleic acids, to cover the entire regions of interest to a tiling depth of at least 2×, preferably at least 4×, more preferably at least 8× (e.g. 10× or more). By mixing a large number of the microbeads, a substantially uniform distribution of individual hybridisation probes throughout the solid support can be achieved, while maintaining a substantially equal distribution of the different hybridisation probes throughout the solid support.

Accordingly, the invention also relates to a library comprising a first microbead and a second microbead, wherein said first microbead is linked to a plurality of hybridisation probes which bind to a first region in a genome of an organism, and wherein said second microbead is linked to a plurality of hybridisation probes which bind to a second region in the genome. The nucleic acid sequence of the first region and the nucleic acid sequence of the second region are typically different from each other. The first region and the second region may be associated with a disease or disorder that affects said organism. For example, the first region and the second region may be located in different oncogenes. Alternatively, the first region and the second region may be located in different coding or non-coding regions (e.g. genes including promoter regions, introns and exons) that each relate to a constitutional disorder or genetic disease, e.g. a hereditary metabolic disorder, to enable the use of the library in pre-natal diagnostics. Typically, a library may comprise more than two microbeads binding to more than two different regions of a genome. For example, the library may comprise at least 10 different microbeads, preferably at least 20, more preferably at least 50 (e.g. more than 100 microbeads). The hybridisation probes on a microbead may all have the same sequence, or they may all have different sequences.

In some embodiment, in place of microbeads which are spherical in shape, the solid support can be formed by microparticles that are polyhedral in shape and approximate the shape of a sphere (e.g. dodecahedron, icosidodecahedron, rhombic triacontahedron etc.).

### Microbead Bed

In some embodiments, the solid support is formed by tightly packing hundreds, thousands, tens of thousands or more microbeads into a microbead bed in a confined space (e.g. by packing the beads into a channel of a column or microfluidic device such that they fill the cross-section of the channel completely and form a microbead bed through which any fluid applied to the channel has to travel). For example, the microbead bed can be formed by loading a suspension of microbeads linked to hybridisation probes into the inner channel of a column that is obstructed with e.g. a porous frit at some point along its length. In some embodiments, the pores of the frit are smaller than the microbeads, so the fluid flow packs the microbeads into a tight bed against the frit. In other embodiments, the diameter of the pores of the frit can be larger than the average diameter of a microbead forming the microbead bed as long as the frit prevents the microbeads from exiting the column. For example, the diameter of the pores of the frit may be 40 to 300% larger than the average diameter of the microbead in the microbead bed. It has been shown experimentally that frits with these larger pore sizes are able to retain the microbead bed while maintaining flow rates of 5-100 μl/min. Packing the microbeads against the frit creates narrow, micron-sized voids between the microbeads that permit fluid to flow between them. The voids are interconnected with the microfluidic channel in which the microbead bed is formed. The narrow paths or voids between the microbeads accelerate the hybridisation process by forcing the nucleic acid molecules in a sample into close contact with the hybridisation probes and increasing the rate of nucleation reactions (i.e. the rate at which the first few base pairs are formed).

In order to minimise unspecific binding, the frit material has a low DNA binding capacity. Polypropylene, in particular polypropylene free of any surface coating, has low DNA binding capacity. Ideally, it withstands rapid changes in temperature during hybridisation. A suitable frit material operates in a temperature range from 20-100° C. Typically, the frit material withstands temperatures of up to 100° C. (e.g. 95° C. or 99° C.). A suitable frit material is autoclavable if it withstands high pressure saturated steam at 121° C. for around 15-20 minutes. The frit material withstands the various chemicals present in the sample and wash buffers and in the elution buffer. In particular, the frit material has chemical compatibility with 95% ethanol and 0.1N NaOH. Preferred frit materials are polyethylene (e.g. ultra-high-molecular-weight polyethylene or high-density polyethylene), polypropylene or glass (e.g. borosilicate). A silica-based frit material may also be suitable for practising the invention. The frit material may be chemically modified to render it hydrophilic. A hydrophilic frit material is preferable because it aids wetting. A suitable hydrophilic frit material includes polytetrafluoroethylene. Polyethylene or polypropylene sintered porous plastic materials (e.g. BioVyon™) are particularly suitable as frit material and are available in pore sizes from 5 to 100 μm (mean flow pore).

In some embodiments, pressure is applied to drive a sample through the microbead bed. Typically, the pressure is between 10 and 500 mbar, e.g. between 25-300 mbar. Preferably, the pressure applied to the microbead bed is between 40 and 250 mbar. For example, during hybridisation, the pressure may be between 40-100 mbar, preferably between 40 and 60 mbar or about 50 mbar. The pressure during washing is typically higher than during the hybridisation step(s), for example, the pressure during washing may be between 100 and 500 mbar, preferably, between 150 and 300 mbar or about 200 mbar. The frit holding the microbead bed in place is chosen to allow flow rates of 5-100 μl/min at the desired pressure. Preferred flow rates are in the range of 25-80 μl/min, e.g. 30-70 μl/min. The flow rate is determined by the thickness of the frit and the average pore size. The pore size has to be compatible with the size of the microbead. The inventors have found that a frit material with pore sizes of 7-20 μm is sufficient to retain beads with an average diameter of 5 μm and above. Typically, the frit will have a thickness of between 1 mm and 4 mm. The average volume porosity of a suitable frit is 20%-50%, preferably 25-40% or about 30%. A suitable frit prevents water applied to the microbead bed from flowing out of the column in the absence of pressure, i.e. no gravity flow will occur and the sample will not drip out of the column when no pressure is applied to the microbead bed.

A column suitable for the preparation of a microbead bed is shown in FIG. 7. A suitable column (1) comprises an inner channel (2) and a porous frit (3), wherein said inner channel (2) is stepwise tapered, dividing the column into a top section (4), a neck section (5) and a bottom section (6), wherein the frit (3) is located in the bottom section (6) of the column (1) and obstructs the entire cross-section of the inner channel (2). The inner channel (2) may taper in the neck section (5) to about 30% to about 50% of the diameter of the inner channel (2) at the top section (4) of the column (1). The inner channel (2) may taper further in the bottom section (6) to about 20% to about 30% of the diameter of the inner channel (2) at the top section (4) of the column (1). In some embodiments, the top section (4) is divided into an upper top section and a lower top section by a rim (8) protruding into the inner channel (2). In certain embodiments, the bottom section (6) has a volume of about 10 μl to about 30 μl (e.g. about 20 μl). The top section (4) can make up about 60-75% of the total length of the column (1). The neck section (5) can make up about 5-10% of the total length of the column (1). The bottom section (6) can make up about 20-30% of the total length of the column (1).

The shape of the column's inner channel, in which the microbead bed is formed, may be specifically adapted to form a microbead bed of consistent thickness and pore size. In this context, it is important that any pipette tip used for adding the microbead suspension on top of the frit is guided to the centre of the inner channel at a sufficient distance from the surface of the frit to allow the full volume of the suspension necessary for the formation of the microbead bed to be expelled from the pipette tip without the empty tip touching the suspension. This can be achieved by tapering the inner channel in such a way that full insertion of the pipette tip is prevented and the tapering locates the tip in the centre of the channel at a sufficient distance from the frit.

The inner channel of the column in which the microbead bed is located may be shaped so as to minimise disturbances to the microbead bed during sample application and washing steps. This can be achieved by, for example, tapering the column's inner channel so that a pipette tip inserted into the inner channel is guided to the centre of the column at a distance sufficiently far away from the microbead bed to minimise any disturbance of it (typically 2-5 mm). As an alternative, a flange may be added at the top of the column (which typically is removable, e.g. in form of a cap that is attached to the top of the column by a hinge). The flange narrows the diameter of the channel at the top so that a pipette tip inserted into the column is guided to the centre of the column's inner channel and blocks the tip from entering the inner channel of the column to maintain the tip at a distance sufficiently far away from the microbead bed to minimises any disturbance of it.

In some embodiments, a rim may be added to the top half or top third of the inner channel of the column (see FIG. 7). The rim is wide enough to rest a pipette tip against it without obstructing the outlet of the tip. Its purpose is to guide pipette tips to the wall of the column so that wash buffer can be pipetted down along the side of the inner channel so as to minimise any disturbance of the microbead bed during washing steps. In addition, a microbead suspension can be applied to the column by resting the pipette tip containing the suspension against the rim, which may aid the formation of the microbead bed because the suspension flows along the wall of the column's inner channel and fills the bottom of the column without the formation of bubbles which, if embedded in the suspension, could interfere with the formation of a suitable microbead bed. The rim may be combined with a flange at the top of the column, where the flange prevents insertion of a pipette tip into the column beyond the rim.

In further embodiments, the bottom section of the column (including the inner channel), where fluids applied to the column exit the inner channel of the column, is further tapered so that the inner channel's smallest diameter is found at the bottom of the column. Adapting the shape of the bottom section of the column allows better dripping and prevents “hanging drops” at the bottom of the column which could contaminate neighbouring samples, if the drops on neighbouring columns are large enough to come into contact (e.g. in an array of columns placed over a 96-well plate), or a microfluidic device in which the column is inserted, e.g. during the removal of the column from the device. In addition, foaming is reduced. Ideally, the bottom section of the column is shaped like a pipette tip to allow for the exact placement of fluid exiting the inner channel into e.g. 96-well plates located under an array of columns, without any cross-contamination of neighbouring wells. In some embodiment, the column wall is thinner at the tip of the bottom section of the column than in other parts of the column, similar to a pipette tip, e.g. to further reduce foaming and the formation of “hanging drops”.

Preferably, the largest diameter of a column is such that an array of 96 columns can be placed on top of a 96-well plate and the tip of each column aligns with the centre of a corresponding well on the 96-well plate, preferably without any of the columns touching each other. In some embodiments, the columns are arranged into arrays for use with a microfluidic device of the invention.

The bottom section of the column also accommodates the frit, which forms the lower boundary of the microbead bed. Typically, the bottom section of the column holds the frit. Tapering the bottom section of the column allows for a tight fit of the frit and thus prevents leakage of beads around the rim of the frit.

In some embodiments, the column has an external flange at the widest diameter of its cross-section. Adding an external flange prevents a pressure bypass when the column is used with the microfluidic device described below. As an alternative, the outside of the column tapers (preferably reflecting the tapering of the inner channel as described above). Tapering of the column may be sufficient to provide a friction fit, so that when the column is placed in a heating element or heating block with bores that are tapered correspondingly to the external shape of the column a seal is created between the column and the heating block. Preferably, the outside of the column has no ribs so that the tapered shape of the column can create a diametrical interference between the column and the heating block.

Representative column designs are shown in FIGS. 6 and 7.

Columns with suitable microbead beds for functionalization with hybridisation probes are commercially available (see reference 11). Typically, however, functionalization with hybridisation probes will take place before forming the microbead bed.

### Hybridisation Probes

The hybridisation probes contain nucleic acid sequences complementary to the sequences of interest in the nucleic acid molecules which are targeted for enrichment. The hybridisation probe is sufficiently long to impart specificity for the binding of a particular target sequence of interest. The hybridisation probes can be 10-500-mer oligonucleotides. Preferably, the hybridisation probes are 20-250-mer oligonucleotides, and more preferably they are 30-150 bases in length. Particularly suitable hybridisation probes have been found to be 80-120mers, and typically hybridisation probes may have a length of 100-140 bases or about 120 bases. Hybridisation probes comprising ≧25 bases, preferably ≧50 bases are preferred. Probes having a length of <500 bases, preferably <400 bases, more preferably <300 bases, are preferred. A typical hybridisation probe is a 50-250-mer oligonucleotide (e.g. 150-200-mer oligonucleotide).

In some embodiments, much larger nucleic acid molecules (>10,000 bases in length) are used as hybridisation probes. For example, bacterial artificial chromosomes (BAC) and yeast artificial chromosomes may be used as hybridisation probes in some applications, e.g. to isolate an entire chromosome from a sample for further downstream analysis. BACs may contain large DNA inserts in excess of 100-175 kb in length.

BACs have been employed as probes for fluorescence in situ hybridisation (FISH). One application of this technology, sometimes referred to as BACs-on-Beads (BoBs) is molecular karyotyping for the detection of chromosome abnormalities (see references 12 and 13). Following isolation from a sample, genomic sample DNA is mixed with reference DNA and then labelled and purified, prior to hybridisation to BACs coupled to beads. Following hybridisation and washing, the ratio of reference to sample hybridised to specific BACS is detected. The hybridisation time in solution for such assays is typically 16-20 hours.

The current invention significantly improves on this method by reducing the hybridisation time by more than an order of magnitude, making it feasible to perform the assay within a working day. This is achieved by attachment of BACs to a solid support and employing the BAC probes as hybridisation probes as described herein. This enables the isolation of target fragments, facilitating the detection of chromosome abnormalities such as insertions, deletions, microdeletions, translocations, aneuploidy, copy number variations (CNVs) and other chromosomal rearrangements. Such genomic events may be missed using shorter oligonucleotide probes [14]. It has also been reported that the use of oligonucleotides for high resolution molecular karyotyping can result in an increase in the detection of CNVs with an uncertain contribution to phenotype [15].

The shorter the oligonucleotide used as hybridisation probe, the longer the unbound area of a hybridised single-stranded nucleic acid molecule of interest that is free to bind to any complement in the sample. To reduce the available unbound area, the length of the hybridisation probe advantageously is ≧40% (e.g. from 50% to 60%) of the average fragment length of the nucleic acid molecules in the sample. However, for most applications, the length of the hybridisation probe is ≦30% of the average fragment length of the nucleic acid molecules in the sample.

The hybridisation probes attached to the solid support are designed to work optimally with a sample that contains nucleic acid molecules of a specified average length. The average length of the nucleic acid molecules is typically specified by the requirements for any applications downstream to the enrichment process. For example, if the enriched nucleic acids are used for sequencing, the read length of the sequencing method of choice will determine the average length of the nucleic acid molecules applied to the solid support.

The length of a hybridisation probe is typically designed to be ≧40%, ≧50% or ≧60% of the average fragment length of a nucleic acid molecule in the mixture. For example, if the hybridisation probes have an average length of 60 bases, the microfluidic device is particularly well suited for the enrichment of nucleic acids having a length of 100 to 150 bases. Alternatively, the hybridisation probes on the solid support are designed to be longer than the average nucleic acid molecules in the mixture that is applied to the microfluidic device for enrichment. The hybridisation probes may be ≧10 bases, preferably ≧20, more preferably ≧50 bases longer than the average fragment length of a nucleic acid molecule in the mixture. For example, where the hybridisation column is used to enrich samples for nucleic acid molecules of interest having an average size of 150 bases, the hybridisation probes may be 160 to 200 bases in length. Having hybridisation probes that are longer than the nucleic acid molecules in the sample applied to the solid support can particularly advantageous when single-stranded adapter molecules are ligated in situ to the hybridised nucleic acid molecules (see below).

To link the hybridisation probes to the solid support, the hybridisation probes preferably contain a functional group, but the addition of a functional group is not always required. The functional group is chosen for its ability to chemically react with the functionalised solid support. In some embodiments, the hybridisation probes are covalently attached to a linker group which contains a functional group, but linkage can also be direct via the functional group. For example, a 5′ amine modification can be used to link a hybridisation group to a NHS-functionalised solid support. In other embodiments, the hybridisation probes are linked to the solid support via a high-affinity, non-covalent linker group, which is covalently attached to the hybridisation probe. One example for such a functional group or linker group is biotin, which can be used with a solid support that has been functionalised with streptavidin. Generally, all hybridisation probes are linked to the solid support via the same functional group.

Typically, all hybridisation probes on the solid support have the same or about the same length. For example, the average hybridisation probe may be 180-mer oligonucleotide, but the solid support may contain a mixture of individual hybridisation probes, which can include e.g. 178-mer oligonucleotides, 179-mer oligonucleotides, 180-mer oligonucleotides, 181-mer oligonucleotides, 182-mer oligonucleotides, etc. This increases the likelihoods that individual probes in a mixture of hybridisation probes are linked with the same efficiency to the solid support so that none of the individual probes is overrepresented on the surface of the solid support. Preferably, all hybridisation probes will have exactly the same length (i.e. be composed of the same number of nucleotides) and contain the same functional group.

For some probe design strategies where physiochemical properties such as the melting temperatures (Tₘ) of probes are matched rather than their length, the hybridisation probes may differ in length. In such an embodiment, the length of individual hybridisation probes may range e.g. from 20-250 bases, preferably 30-150 bases, more preferably between 80-120 bases.

Whereas the number of hybridisation probes that can be fitted on a microarray is limited both by the available surface area and the cost involved, such limitations do not exist to the same extent when the solid support of the invention is used for enrichment because the hybridisation probes are randomly attached to the entire surface of a functionalised solid support, without any need for special equipment to place the probes on fixed positions. Therefore a high probe density of the entire surface of the solid support can easily and cheaply be achieved using methods well-known in the art. The number of hybridisation probes can also be increased relatively easily due to the much larger surface area available on a porous solid support. For example, optimal surface-hybridisation probe density for maximum nucleic acid hybridization efficiency have been observed in the prior art at hybridisation probe densities in the range of about 1×10¹² to about 9×10¹² probe molecules/cm² [4]. Where the solid support is a microbead bed, the probe density can range from 1 amol/mg microbead to 1 μmol/mg microbead. Typically, the probe density is between 0.01 pmol/mg and 2500 pmol/mg, preferably between 0.1 pmol/mg and 100 pmol/mg or between 0.1 pmol/mg and 10 pmol/mg.

Hybridisation efficiency requires a driver. The driver can either be excess input nucleic acids in the sample or excess hybridisation probes on the solid support. Capture efficiency can be increased by providing a solid support with a hybridisation probe density such that the number of individual hybridisation probes complementary to a specific sequence of interest exceeds the theoretical number of nucleic acid molecules that carry this specific sequence in a typical sample applied to the solid support. For example, if a typical sample contains 100 ng of DNA so that about 1,000,000 copies of a specific DNA sequence of interest are presented in the sample, then the solid support for analysing the sample should have a probe densities that allows for e.g. a 10-fold or 100-fold excess of the probe which is complementary to the specific DNA sequence of interest. Under these conditions, the excess probe concentration can drive hybridisation.

A single hybridisation probe can be used to target a nucleic acid molecule of interest. However, more typically, more than one hybridisation probe is used to target a nucleic acid molecule of interest. Typically, sample preparation results in random fragments of a target nucleic acid molecule. Therefore to increase the likelihood that a nucleic acid molecule of interest is completely represented, multiple probes are necessary.

To increase the likelihood that a sequence of interest is completely represented, it may be preferred that hybridisation probes for a particular target territory overlap with each other. For example, hybridisation probes may be designed to tile a target territory to a depth of 2×, more preferably to a depth of 3×, even more preferably to a depth of 4×, 8× or 12× (for details see e.g. reference 16). In some embodiments, the total combined length of hybridisation probes expressed in bases exceeds the total combined length of the target sequence by 10%, more preferably 20%, even more preferably 30%.

In order to increase the likelihood of equal representation of nucleic acid molecules of interest that originate from the centre of a target territory and from the boundaries of a target territory, the hybridisation probes for a particular target territory are designed to extend beyond the boundaries by 10-100 bases, e.g. by 20 bases, preferably 40 bases, more preferably 60 bases. For example, if the target territory is a particular exon of a gene, some of the hybridisation probes covering the target territory extend into the intron region e.g. by 25 bp.

### Hybridisation Conditions

The hybridisation probes bind to the nucleic acid molecules of interest in a sample under stringent hybridisation conditions. To reduce unspecific binding of nucleic acid molecules which do not contain sequences complementary to the nucleic acid sequences of the hybridisation probes, a blocking step is typically performed prior to the addition of the sample. A blocking buffer containing blocking oligonucleotides is typically used to wash the solid support prior to the addition of the sample. Unspecific interactions of the nucleic acid molecules in the sample with the solid support can further be minimised by the addition of blocking oligonucleotides to the hybridisation buffer. For example, unspecific interactions of sample DNA may be blocked by washing the solid support with hybridisation buffer containing salmon sperm DNA. A typical hybridisation buffer consists of 1×Hi-RPM buffer (Agilent Technologies, Inc.) supplemented with 50 ng/μl salmon sperm DNA.

The flow rate of fluids through the solid support can be in the range of 1 nl/minute to 1 l/minute, but typically in the context of a microfluidic device a flow rate of 1-100 μl/minute, preferably 2-50 μl/minute, more preferably, 4-25 μl/minute is maintained to drive fluids through the solid support. For example, a flow rate of about 5 μl/minute may be used to drive hybridisation buffer, washing buffer and elution buffer through the solid support. Using a constant flow rate for applying the sample to the solid support rather than gravity flow or diffusion may improve hybridisation because each part of the solid support is brought into contact with the sample under the same conditions.

Generally, the flow rate will be chosen to achieve a certain number of volume exchanges within the solid support over a given period of time. For example, 1-10 volume changes per minutes may be suitable to achieve hybridisation, whereas a higher number of volume changes per minute (e.g. 20 or more, preferably 40 or more, more preferably 60 or more volume changes per minute) may be desirable during washing steps. For instance, if the solid support of the invention has a void volume of about 0.5 mm³, then a flow rate of 5 μl/minute through the solid support corresponds to 10 changes of the void volume per minute, and a flow rate of 30 μl/minute corresponds to 60 changes of the void volume per minute. What flow rate is chosen may depend on the concentration of the nucleic acid in the sample and the total sample volume. For example, more volume changes per minute may be desirable to apply a large volume of a relatively dilute sample. The above ranges for volume changes per minute are particularly suitable where the DNA concentration in a sample applied to the solid support of the invention is about 1 ng/μl to about 100 ng/μl.

The mixture containing the nucleic acid molecules of interest is diluted in hybridisation buffer before it is brought into contact with the solid support. Once the sample is in contact with the solid support, the temperature is increased, typically to between 55-65° C., to allow the nucleic acid molecules to anneal to the hybridisation probes. The process of hybridisation can be very fast when the hybridisation probes are linked to a solid support with micron-scale voids because the nucleic acid molecules are prevented from diffusing very far from the hybridisation probes (as is the case in conventional microarrays). The hybridisation step may be performed for a time period of 1 second to 1 hour. However, typical hybridisation times are between 1 and 15 minutes, e.g. 2-10 minutes. Generally a continuous flow of hybridisation buffer or blocking buffer is maintained during the entire process to drive the sample through the solid support. However, it is also possible to e.g. apply the sample to the solid support and then stop the flow to allow for hybridisation or to drive the sample through the solid support in intervals where the period between intervals allows for additional time in which the nucleic acid molecules within the sample can interact with the hybridisation probes on the solid support. Nucleic acid molecules that do not contain complementary sequences remain unbound and are removed by the constant flow of hybridisation buffer. Any non-specifically bound nucleic acid molecules are subsequently removed by flushing the solid support with a stringent wash buffer. Typical compositions of stringent wash buffers are well known to the skilled person. For example, high stringency can be achieved by including 0.1×SSPE and 0.005% (v/v)N-lauroylsarcosine or 0.1% SDS and 0.1×SSC into a buffer.

To further reduce unspecific interactions and increase the efficiency of the washing step, the temperature may be decreased by only about 5-10° C. from the hybridisation or annealing temperature while wash buffer is applied to the solid support. Alternatively the temperature is decreased to 20-30° C. during the washing step.

The wash buffer may be applied at a higher flow rate than the hybridisation buffer or blocking buffer. For example, the flow rate during the washing step may be increased 5-10 fold, e.g. from 5 μl/minute to 30 μl/minute. Wash buffer may be applied continuously, e.g. for 5-30 minutes, or may be applied discontinuously, e.g. in multiple 5-minute intervals with 1-minute pauses in-between each washing step.

After washing, a mixture of nucleic acid molecules is retained on the solid support that is highly enriched for the nucleic acid sequences of interest.

### Elution

Finally, the highly enriched nucleic acid molecules of interest, which remain bound to the hybridisation probes, are eluted, e.g. through a rise in temperature, a decrease in the salt concentration or a combination of both. For example, the temperature of the solid support can be raised to about 90-100° C., preferably to about 95° C., to elute any bound nucleic acids. If elution is initiated by an increase in temperature, the flow of wash buffer may be continued or the flow may be switched to an elution buffer (e.g. TE buffer), which can be used to store the eluted sample.

Alternatively, hybridisation duplexes can be disrupted by physiochemical means such as modification of the pH. For example, 100 mM NaOH can be used to remove any bound nucleic acids from the solid support. For subsequent application, the pH of the elute needs to be adjusted to neutral, e.g. by the addition of Tris-HCl, pH 7.5.

Eluted nucleic acid molecules are collected, e.g. by diverting the outflow of washing buffer from the solid support into a collection tube. To further enrich the sample, the eluted nucleic acid molecules can be added to a solid support for one or more additional rounds of hybridisation (after having been diluted in hybridisation buffer, if necessary). The solid support may be the same solid support that was used during the previous hybridisation steps, or it may be a different solid support. Prior to a further hybridisation step, the eluted nucleic acid molecules may be amplified and optionally purified. As an alternative, the eluted nucleic acid molecules may be amplified and subsequently purified for use in downstream applications such as sequencing. If the nucleic acid molecules lack suitable sequences for amplification, adapters containing the required sequences may be ligated to the eluted nucleic acid molecules.

In some embodiments, the eluted nucleic acids already contain adapter molecules for sequencing. Advantageously, the present invention allows elution using very small volumes (10-50 μl). The eluted and enriched nucleic acid molecules are sufficiently pure to be used directly for downstream applications such as sequencing.

### Joining of Adapter Molecules

For certain downstream applications such as amplification and NGS, it is preferable that the nucleic acid molecules are furnished with adapter nucleic acids which are joined to both ends of each molecule. The adapter molecules can provide priming sites for subsequent amplification by PCR, as well as sequences that are specific to the sequencing method of choice, e.g. sequences that hybridise to the flow-cell surface in an Illumina® sequencer. For example, the 5′ adapter can contain the forward primer site for downstream amplification and sequencing, while the 3′ adapter molecule can contain the reverse primer site.

Typically, these sequences are joined to fragmented nucleic acid molecules before enrichment so that all fragments possess adapter sequences, therefore avoiding the need for further ligation steps after enrichment. The inventors found that ligating adapter nucleic acids for indexing and sequencing to the target nucleic acid molecules after enrichment can reduce the amount of reagents that is required and can further accelerate the preparation of enriched nucleic acid molecules for sequencing, in particular when the adapter nucleic acids are added to the flow through the solid support of a microfluidic device.

In some embodiments of the invention, the highly enriched mixture of nucleic acid molecules of interest that is eluted from the solid support is ligated to adapter molecules that provide the required binding site for downstream applications such as amplification and/or sequencing.

Alternatively, nucleic acid molecules can be ligated with the adapter nucleic acids while they are hybridised to the hybridisation probes of the solid support (in situ). In situ modification of hybridised DNA, including ligation, has been demonstrated previously and is described in reference 17.

After the ligation reaction, the single-stranded adapter terminated nucleic acid molecules are eluted from the solid support through a rise in temperature, a decrease in the salt concentration or a combination of both.

Adapter ligation to the 3′ ends and the 5′ ends of the nucleic acid molecules can occur either simultaneously or sequentially. Generally, the ligation reaction is catalysed by a ligase that exclusively recognises double-stranded nucleic acid sequences, typically a DNA ligase. Adapter ligation to the 3′ ends and adapter ligation to the 5′ ends of the nucleic acid molecules preferably occur sequentially (i.e. in separate steps). This has the advantage that the production of undesired ligation products is reduced. In addition, the sequencing primers can be changed or replenished separately, for example by being located in separate cartridges of a microfluidic device.

For example, when the ligation steps are sequential, mixture of a 5′ adapter and a ligase may be added first. The order can be reversed and the 3′ adapter can be ligated to the nucleic acid molecules before ligation of the 5′ adapter.

For example, the 5′ adapter may hybridise to the hybridisation probe (e.g. if in situ hybridisation is used and the hybridisation probe is longer than the average nucleic acid molecule in the sample) or the nucleic acid molecule (e.g. if a duplex adapter is ligated to single stranded nucleic acid molecules), and the nick between the 5′ end of the nucleic acid molecule and the 3′ end of the 5′ adapter is ligated by the ligase. After a washing step, a mixture of a 3′ adapter and a ligase is added. The 3′ adapter also hybridises either to the hybridisation probe (e.g. if in situ hybridisation is used and the hybridisation probe is longer than the average nucleic acid molecule in the sample) or the nucleic acid molecule (e.g. if a duplex adapter is ligated to single stranded nucleic acid molecules), and the nick between the 3′ end of the nucleic acid molecule and the 5′ end of the 3′ adapter is ligated by the ligase. After a further washing step, the single-stranded, adapter-terminated nucleic acid molecules can be recovered for downstream applications (e.g. amplification and sequencing).

Alternatively, adapter ligation to both the 3′ ends and the 5′ ends of the nucleic acid molecules can be performed in a single step. In this embodiment, a mixture comprising a 5′ adapter, a 3′ adapter and a ligase are added to the nucleic acid molecules. The nicks between the ends of the nucleic acid molecules and the adapters are joined by ligation. After a washing step to remove the ligation mixture, the single-stranded, adapter-terminated nucleic acid molecules can be recovered for downstream applications (e.g. amplification and sequencing).

Alternative methods to ligation exist for joining nucleic acids together and could be used to modify the foregoing embodiments. For example, addition of adapter molecules using enzymes such as transposases have been successfully employed [18].

### Suitable Adapter Molecules

Suitable adapter molecules can be either single-stranded or double-stranded. For in situ ligation, the choice of adapter molecules may depend on the length of the hybridisation probes bound to the solid support relative to the length for the nucleic acid molecules in the mixture that is added to the solid support.

Preferably, the adapter molecules include terminal blocking groups (e.g. C3 spacers or zig-zag blocks) so that only one end of an adapter molecule is susceptible to ligation. This can reduce the production of undesired ligation products.

### Single-Stranded Adapter Molecules

In one embodiment of the invention, the hybridisation probes on the solid support are longer than the hybridised nucleic acid molecules. In this embodiment, a mixture of single-stranded adapter molecules containing short stretches of degenerate bases on one end can be used to hybridise to the hybridisation probes on the solid support. Only a small subset of these adapter molecules will bind immediately adjacent to a hybridised nucleic acid molecule. Once adapter molecules have hybridised to a probe on either side of a hybridised nucleic acid molecule, a ligase that exclusively recognises double-stranded nucleic acid sequences can join the ends of the respective 5′ and 3′ adapter molecules to the respective 3′ end and 5′ ends of the hybridised nucleic acid molecule.

By locating the short stretch of degenerate bases on one end of the adapter molecule and a blocking group on the opposite of the adapter molecule, a ligation reaction with a ligase that exclusively recognises double-stranded nucleic acid sequences occurs only when the adapter molecule has hybridised to the hybridisation probe directly adjacent to a hybridised nucleic acid molecule.

### Double-Stranded Adapter Molecules

In one embodiment, double-stranded adapter molecules are composed of two oligonucleotides that form a double-stranded or duplex region. These adapter molecules are also referred to as adapter duplexes. One of the oligonucleotides further comprises a single-stranded overhang region formed by a short stretch of degenerate bases via which the adapter duplexes can hybridise to the ends of single-stranded nucleic acid molecules. The ends of both oligonucleotides in the adapter complex opposite to the overhang are typically terminated by blocking groups.

Double stranded adapter molecules may be hybridised to nucleic acid molecules in situ on a solid support, in which case the hybridisation probes on the solid support must be shorter than the hybridised nucleic acid molecules.

Typically, the stretch of degenerate bases is terminated by a blocking group so that ligation can occur only when the adapter duplex is hybridised to the nucleic acid molecule. In addition, the ligation reaction can take place only between the nucleic acid molecule and the oligonucleotide of the adapter complex that lacks the single-stranded overhang.

If in situ ligation is used, the oligonucleotide in each adapter duplex that has not been ligated due to the presence of a terminal blocking group is separated from the single-stranded, adapter-terminated nucleic acid molecule during elution (which can be triggered by e.g. a rise in temperature or a decrease in the salt concentration). Since these oligonucleotides are shorter than the adapter-terminated nucleic acid molecules, they are eluted more quickly from the solid support. The single-stranded, adapter-terminated nucleic acid molecules are eluted more slowly and can be recovered separately for use in downstream applications such as amplification and sequencing.

In another embodiment, double-stranded adapter molecules are composed of a single oligonucleotide that base-pairs with itself to create a short complementary region (stem) and a single-stranded region (loop). These stem-and-loop adapters (also referred to as hairpin adapters) can be used as an alternative to adapter duplexes. One end of the oligonucleotide comprises a single-stranded overhang region formed by a short stretch of degenerate bases via which the hairpin adapter can hybridise to the ends of single-stranded nucleic acid molecules. The stretch of degenerate bases is terminated by a blocking group so that ligation can occur only when the adapter duplex is hybridised to the nucleic acid molecule. The advantage of using a single oligonucleotide as adapter is that only one blocking group is required which is used to terminate the stretch of degenerate bases to avoid undesired ligation products.

### Downstream Applications

Once nucleic acid molecules have been enriched and tagged with suitable adapter sequences, the adapter-terminated nucleic acid molecules can be amplified, e.g. by using conventional PCR, and/or sequenced, preferably using next-generation sequencing (NGS). In this context, NGS refers to any technology which enables sequencing by methods other than DNA sequencing with irreversible chain-terminating inhibitors, also referred to as Sanger sequencing [19], or Maxam-Gilbert sequencing [20].

Other modification of the nucleic acid molecules may be performed prior to downstream applications. For example, methylated DNA may be bisulphite-treated after the enrichment process to enable bisulfate sequencing.

NGS covers a variety of sequencing technologies such as “massively parallel signature sequencing” (MPSS), polony sequencing, pyrosequencing, cluster sequencing or sequencing-by-synthesis (also referred to as Illumina or Solexa sequencing), and sequencing-by-ligation (e.g. SOLiD sequencing), single-molecule real-time (SMRT) sequencing, sequencing by incorporating reversible terminator nucleotides, nanopore sequencing (e.g. hemolysin nanopore sequencing), and sequencing by the detection of hydrogen ions released during nucleotide incorporation (Ion Torrent) etc. (see references 21 and 22)

Two key benefits are offered by NGS. Firstly, large numbers of genes and variants can be screened in parallel—anything from a single gene to a whole genome. The second key benefit of NGS is scalability—the ability to test high numbers of targets in large numbers of patients, as the demand for molecular testing increases.

Combining the enrichment process of the present invention with certain NGS technologies may be particularly advantageous. For example, the high throughput achieved by cluster sequencing or sequencing-by-synthesis could be further increased by combining it with the enrichment process of the invention, and therefore become a more valuable tool for diagnostic applications. Similarly, combining the enrichment process of the invention with nanopore sequencing could provide significant advantages in terms of speed and costs associates with sequencing large numbers of DNA sequences.

The enrichment method of the invention can be combined with pyrosequencing, polony sequencing, sequencing by incorporating reversible terminator nucleotides, nanopore sequencing, sequencing by ligation, and ion torrent sequencing.

Although whole genome and exome sequencing have diagnostic value, for many diseases, a targeted sequencing approach is typically much more appropriate for diagnostic purposes: reducing incidental findings, reducing the complexity of data analysis, and reducing cost per sample.

For many inherited diseases, a very high diagnostic yield can be achieved with a small panel of clinically-actionable genes and variants. For cancer, smaller panels enable ultra-high depth sequencing for detection of low frequency mutations. Typically, a panel consists of 5-20 genes, but in some instances larger panels consisting of e.g. 30, 50 or 100 genes may be preferred. Rather than targeting entire genes, regions can be targeted including “hot exons”, “hot spots” and non-coding sequences. Genes and regions should be considered as being interchangeable in the context of the present disclosure.

### Microfluidic Device

The process of enrichment by hybridisation is advantageously performed in a purpose-built microfluidic device. The microfluidic device comprises a channel into which a solid support is packed. The solid support comprises a plurality of interconnected, micron-sized voids that permit fluids to flow between them. A plurality of hybridisation probes is bound to the surfaces which form the voids.

Preferably, the channel forms part of a hybridisation column which can be inserted and removed from the microfluidic device. This is advantageous because it allows the device to be repurposed for different enrichment applications by simply exchanging the hybridisation column. In some embodiments, the microfluidic device can accommodate more than one hybridisation column, e.g. 6, 8, 12, 24, 48, 96 or 384 hybridisation columns. For example, the device may be set up to hold an array of 96 or 384 hybridisation columns which are arranged in a 2:3 rectangular matrix. As an alternative, the device may be set up to hold an array of 8 or 12 columns. The hybridisation columns may be provided either individually or as pre-arranged arrays (e.g. in form of strips or plates that contain multiple column units that are connected to each other). For instance, hybridisation columns may be provided as strips of 8 or 12 columns or as break-away plates of 8×12 columns that break of as 8 or 12 column strips, or as plates of 96 or 384 column arrays. The various embodiments described below in relation to a simple hybridisation column can easily be adapted to accommodate a multi-column set-up.

Typically, the channel is positioned within a temperature control element (e.g. a heating element or heating block with one or more bores in which one or more hybridisation columns can be inserted) and a temperature sensor to allow for precise control of the temperature of the fluid within the channel. Preferably, the heating element includes a Peltier element to achieve rapid changes in temperature.

In some embodiments, the channel is connected to a reservoir. The reservoir can be sealed so that the reservoir can be pressurised with a source of compressed gas (e.g. nitrogen), which is connected to the reservoir via a tubing. Any fluid within the channel is driven through the solid support by the resulting pressure gradient between the reservoir and the downstream channel. A valve within the tubing which connects the gas supply to the reservoir can be opened and closed by a controller, which can control the flow rate of the fluid which is driven through the channel. For example, the device may include an electronic controller, which can be programmed to control the flow rate through the channel of the hybridisation column. In some embodiment, a pressure sensor is located in the reservoir to measure the pressure in the reservoir.

A set-up with pumping means to drive a fluid through the channel allows control over the flow rate of the fluid through the solid support. Means other than those described in the preceding paragraph for pumping fluids through the solid support (such as a syringe pump or a peristaltic or diaphragm pump suitable for microfluidic applications, or alternatively a suction device) are readily apparent to the skilled person. Typically, the pump, or any other suitable pressure-gradient generating device, creates a pressure of between 10 and 500 mbar, e.g. between 25-300 mbar, within the reservoir. Preferably, the pressure applied to the microbead bed is adjusted during operation, e.g. by means of an electronic pressure regulator, resulting in different flow rates. A suitable operating range is between 40 and 250 mbar. For example, during hybridisation, the pressure may be adjusted to between 40-100 mbar, preferably to between 40 and 60 mbar or about 50 mbar. A pressure of about 50 mbar has been found to be sufficiently high to prevent gas formation at the temperatures encountered during sample denaturation and hybridisation (95° C. and 65° C.). The pressure during washing is typically higher than during the hybridisation step(s), for example, the pressure during washing may be adjusted to between 100 and 500 mbar, preferably, to between 150 and 300 mbar or about 200 mbar. For example, the flow rate of a fluid through the solid support can be adjusted from 50 nl/minute to 5 ml/minute. More typically, the flow rate can be adjusted to 1-100 μl/minute, preferably 2-50 μl/minute, more preferably, 4-25 μl/minute is maintained to drive fluids through the solid support. Suitable flow rates include 25-80 μl/min, e.g. 30-70 μl/min. For example, a flow rate of about 5 μl/minute may be used to drive hybridisation buffer, wash buffer and elution buffer through the solid support. Alternatively, the flow rate with which the wash buffer is applied may be 5-10 fold higher than the flow rate with which the hybridisation buffer is applied. For example, the hybridisation buffer may be applied to the solid support at a flow rate of 5 μl/minute, while washing takes place at a flow rate of 30 μl/minute.

To prevent a pressure bypass when the microfluidic device is used with the hybridisation columns described above, the columns are preferably friction-fitted into a heating block. The tight fit of the columns into the heating block prevents air leaking around the columns.

Built-in temperature control and control of the flow rate is advantageous because it allows automation of the process therefore minimising variations in the hybridisation conditions and increasing reproducibility of the enrichment process. In addition, the stringency of the hybridisation process can be tightly controlled therefore improving specificity and selectivity of the enrichment process.

One end of the channel comprising the solid support may be connected to tubing. The tubing can be connected to a collection tube to collect the eluate from the solid support that contains the enriched nucleic acid molecules of interest. In some embodiments, the collection tube passes through a detection chamber. The detection chamber comprises an optic sensor for label-free, real-time detection and, optionally, quantification of nucleic acids within the outflow from the solid support. The presence of detection chamber is particularly advantageous for processes where single-stranded, adapter-terminated nucleic acid molecules need to be distinguished from unligated oligonucleotides of adapter duplexes used for in situ ligation.

In some embodiments of the invention, a sample containing nucleic acid molecules of interest may be applied to the channel and driven through the solid support manually. This simplifies the microfluidic device and reduces the costs. Manual intervention is possible due to the drastically reduced hybridisation times achieved by the present invention. For example, the sample may be applied to the solid support located within the inner channel of the hybridisation column either directly by pipetting the sample into the column or by means of a syringe containing the sample, whereby the syringe is connected to tubing or capillary that is connected to one end of the inner channel of the hybridisation column. Once the syringe's plunger is depressed, the sample will be injected into the tubing or capillary and will be pushed in the channel and through the solid support.

In some embodiments, the microfluidic device is used to prepare the solid support of the invention. For example, an empty column comprising an inner channel which is partially closed on one end (e.g. by the presence of a porous filter, frit or permeable membrane) may be placed into the device. A solid support can be formed in the channel by placing a suspension of microbeads in the channel above the partially closed end. Pressure, e.g. from a source of compressed gas connected to a reservoir upstream of the channel, can be applied to force the suspension through the channel. Alternatively, suction may be used to drive the suspension through the channel Because the microbeads cannot pass the partially closed off part of the channel, they are tightly packed forming a microbead bed. The suspension may contain between 1 μg and 1.5 g of microbeads, however more typically, the amount of microbeads ranges from 0.1 to 100 mg, preferably 0.25 to 10 mg.

Downstream molecular operations, such as the ligation of indexing and sequencing adapters to the nucleic acid molecules, can be performed by adding small volumes of the required reagents to the microfluidic flow through the solid support. This can further accelerate the preparation of enriched nucleic acid molecules that are ready for sequencing.

In some embodiments, the mixture of nucleic acid molecules applied to the microfluidic device already contain the required indexing and sequencing adapters, and the eluted and enriched nucleic acids can be used directly for downstream applications such as sequencing. In a specific embodiment, the microfluidic device of the invention may therefore be coupled to a sequencer.

### General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x is optional and means, for example, x±10%.

### DESCRIPTION OF THE DRAWINGS

FIG. 1: Arrangement of microbeads in a hybridisation column for rapidly enriching DNA fragments for sequencing. (A) Microbeads (circles) are loaded into a plastic column (striped areas) with a porous frit (area filled with wavy lines) at some point along its length. The pores of the frit are smaller than the microbeads, so the fluid flow packs the microbeads into a tight bed against the frit. (B) The packed microbeads have narrow, micron-sized voids between them that permit fluid to flow (arrows) between them. (C) The small channels force DNA molecules in the flow (striped line) to come into close contact with a mixture of oligonucleotide probes on the surfaces of the packed microbeads that are complementary to the targeted DNA (solid lines) or other targets (dotted lines). A scale bar in (C) shows the approximate size of the voids when the microbeads are 5 μm in diameter. Note that the DNA fragment and probes are represented as being 5- to 10-fold longer than they are in reality (the fragments are <300 nucleotides in length and hence <100 nm).

FIG. 2: Process for enriching DNA for sequencing using microfluidic flows to accelerate hybridisation. Microbeads are functionalised with tens, hundreds, or thousands of different oligonucleotides (60-120 nt) targeted against specific regions of a genome, e.g. human, with each microbead displaying a mixture of these probes. The microbeads are loaded into the device and packed into a tight bed by fluid flow. A sample of genomic DNA is fragmented into pieces of 150-200 bp, adapters for sequencing are optionally added, and the DNA is loaded into the device upstream of the packed bed. The DNA sample is driven through the packed bed by a slow flow of hybridisation buffer at a controlled temperature (65° C.). The DNA fragments in the sample that are recognised by the oligonucleotide probes hybridise to the surface of the microbeads and are retained. The narrow channels between the microbeads accelerate this process by forcing the DNA fragments into close contact with the probes and increasing the rate of nucleation reactions. Non-targeted DNA fragments pass through the bed without hybridising to the probes, or only weakly. One or more washing steps where a wash buffer replaces the hybridisation buffer in the flow eliminate any remaining non-targeted DNA. The temperature of this step is adjusted to control the stringency of the wash(es). Finally, the targeted DNA, which is still hybridised to the probes, is eluted by raising the temperature while continuing the flow of wash buffer. This eluted DNA is then collected for sequencing. If the fragments lack adapters, then they need to be added before sequencing.

FIG. 3: Experimental data showing the selective enrichment of one oligonucleotide from a mixture of two. In this run there were 50 pmol of probe and 500 fmol of each labelled oligonucleotide.

FIG. 4: A plot of read depth versus base number through the entire genome of lambda phage. The spike peaking at base 21,986 corresponds to the region of the genome targeted by the microbead-conjugated probe.

FIG. 5: ATM gene coverage using the set-up described in Example 4. Specific, reproducible and uniform enrichment of the target regions is observed.

FIG. 6: Column design illustrating how the tapering of a column used for preparing a microbead bed can aid in achieving a microbead bed of consistent thickness and pore size and minimising any disturbance of the microbead bed during subsequent steps such as addition of the sample and washing of the microbeads. Column dimensions are given in millimetres. Panel A shows a column design with a reservoir on top which tapers into a narrow inner channel to prevent full insertion of a P100 tip, while allowing insertion of a P20 to a level 1-4 mm distant to the microbead bed to load the DNA sample. Panel B shows a column design with a tip guidance feature in a cap placed on top of the column's wide inner channel. The guidance feature contains a flange that centres both P20 and P100 tips above the location of the microbead bed at a distance sufficient to minimise any disturbance of the microbead bed during its formation and during sample loading. In addition, the column design in panel B exhibits a rim in the internal channel of the column about half way down the length of the column. The rim is placed at a sufficient distance from the top of the column so that P200 tips inserted into the guidance feature bottom out at the level where the rim is located. When wash buffer is added to the column using a P200 tip, the rim guides the tip so that the buffer runs down on the side of the wall minimising any disturbance of the microbead bed.

FIG. 7: A schematic drawing of a column suitable for practising the invention. The column (1) comprises a inner channel (2) and a porous frit (3). The inner channel (2) is stepwise tapered, dividing the column (1) into a top section (4), a neck section (5) and a bottom section (6). The frit (3) is located in the bottom section (6) and obstructs the entire cross-section of the inner channel (2). The top section (4) can be fitted with a removable cap (7), which contains an opening in the top that guides pipette tips inserted into the column (1) to the centre of the inner channel (2). The top section (4) is divided into a upper top section and a lower top section by rim (8). The inset on the left shows a rendering of the moulded column.

### MODES FOR CARRYING OUT THE INVENTION

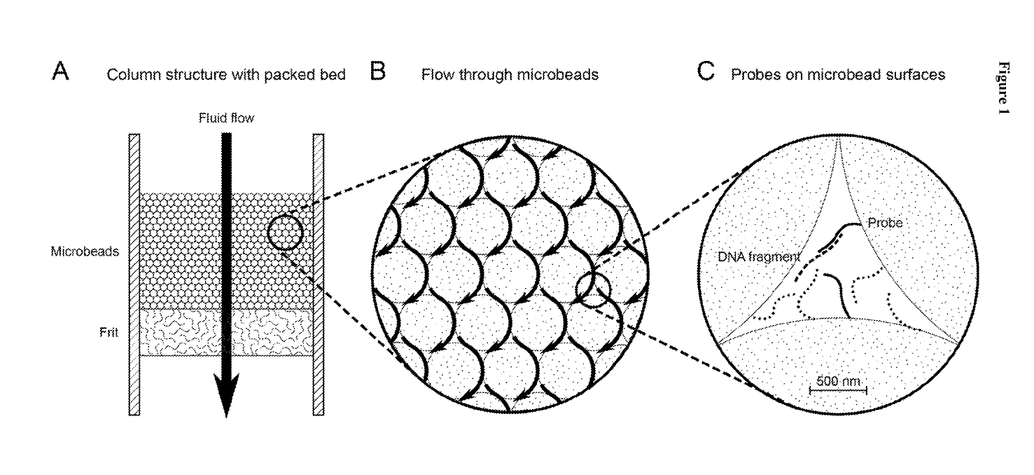

DNA molecules of interest can be enriched rapidly by passing a DNA sample through a packed bed of microbeads which are conjugated with hybridisation probes against specific sequences in the DNA molecules of interest (FIG. 1). The constrained diffusion length of the microbead bed enables the targeted DNA to be captured and enriched relative to the non-targeted DNA in a matter of minutes rather than hours.

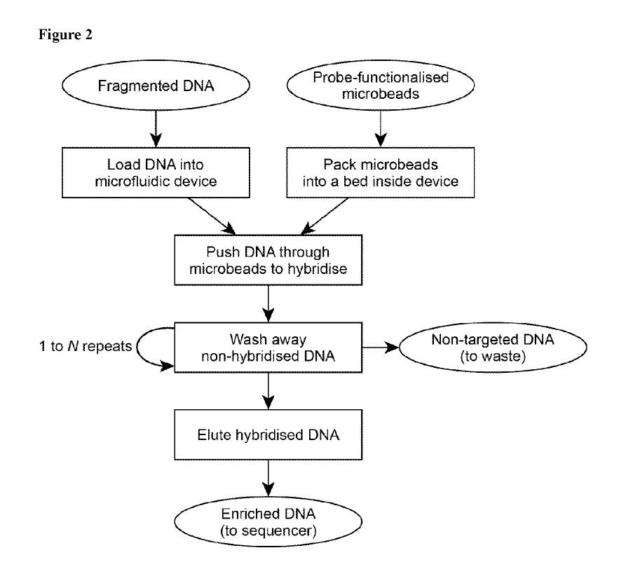

An overview of the workflow for enriching DNA molecules of interest in a DNA sample is provided in FIG. 2. The microbeads are functionalised with tens, hundreds, or thousands of different oligonucleotides (typically 60-120mers) targeted against specific regions of a genome, e.g. the human genome, with each microbead displaying a mixture of these probes. The microbeads are loaded into a microfluidic device and packed into a tight bed by fluid flow.

A sample of genomic DNA is fragmented into pieces of 150-200 bp, adapters for sequencing are optionally added, and the DNA is loaded into the device upstream of the packed bed. The DNA sample is driven through the packed bed by a slow flow of hybridisation buffer at a controlled temperature (65° C.). The DNA fragments in the sample that are recognised by the oligonucleotide probes hybridise to the surface of the microbeads and are retained. Non-targeted DNA fragments pass through the bed, binding to the probes only weakly or not all.

One or more washing steps where a wash buffer replaces the hybridisation buffer in the flow eliminate any remaining non-targeted DNA. The temperature of this step is adjusted to control the stringency of the wash(es).

Finally, the targeted DNA, which is still hybridised to the probes, is eluted by raising the temperature while continuing the flow of wash buffer. This eluted DNA is then collected for downstream applications such as sequencing. If the fragments lack adapters, then they need to be added before sequencing.

### Example 1: Rapid Enrichment of a Fluorescently-Labelled Oligonucleotide

To demonstrate and quantify the rapid enrichment of a specific sequence in a mixture of nucleic acids, two fluorescently-labelled oligonucleotides were combined and passed through a bed of microbeads functionalised with a probe complementary to one of them.

### Preparation of Probe-Functionalised Microbeads

1. A 60-mer oligonucleotide probe with a 5′ amine modification (FB-AmC6-ProbeA: 5′-TGAGGCTTGC ATAATGGCAT TCAGAATGAG TGAACAACCA CGGACCATAA AAATTTATAA-3′ (SEQ ID NO: 1); Integrated DNA Technologies, Inc.) was synthesised and diluted to 1 μM concentration in nuclease-free water.
2. 5 μm-diameter NHS-functionalised silica microbeads (Bioclone, Inc.) were resuspended in 2× Suspension Buffer (Bioclone, Inc.) to a density of 200 g/1.
3. 100 μl of the oligonucleotide solution were combined with 100 μl of the microbead suspension in a 1.5 ml microcentrifuge tube. The coupling reaction was, therefore, performed at a probe density of 5 picomoles per milligram of microbeads.
4. The tube was mounted on the rotisserie in a rotating hybridisation oven and incubated at 50° C. for 16 hours with constant rotation.
5. A magnetic rack was used to separate the microbeads from the supernatant, which was discarded.
6. The microbeads were resuspended in 200 μl 1×Washing Buffer (Bioclone, Inc.), vortexed, and placed back on the magnetic rack. Once again, the supernatant was discarded. The steps constituted one ‘wash’.
7. The microbeads were washed a further two times in 200 μl 1× Washing Buffer (Bioclone, Inc.) and twice in 500 μl nuclease-free water at 99° C.
8. The microbeads were resuspended at 20 mg/ml density in a storage buffer containing 250 mM Tris-HCl, pH 7.6, 20 mM EDTA, 0.1% Tween-20, and 0.02% sodium azide (all Sigma-Aldrich Co. LLC).

The above steps were repeated with a 10 μM solution of FB-AmC6-ProbeA in step 1 so that the microbeads were coupled with a 10-fold greater density of probe (50 pmol/mg).

### Enrichment

1. 1 mg of the probe-functionalised microbeads was loaded into a PEEK capillary with an internal bore of 381 μm (IDEX Corp.). A 0.5 μm-pore PEEK filter (IDEX Corp.) at the end of the capillary trapped the microbeads, causing them to pack into bed with a diameter of 381 μm and a length of about 10 mm
2. Hybridisation buffer was prepared as 1× Hi-RPM buffer (Agilent Technologies, Inc.)

supplemented with 50 ng/μl salmon sperm DNA (Life Technologies). The capillary was heated to 65° C. in a waterbath and the hybridisation buffer was pumped though the microbead bed at 10 μl/minute for 10 minutes using a Mitos Duo XS-Pump (Dolomite Ltd.). This incubation served to pre-hybridise (block) the microbeads and other surfaces inside the capillary.
3. A 9725i HPLC manual injection valve (IDEX Corp.) was used to introduce a 5 μl pulse of fluorescently-labelled 100-mer oligonucleotides dissolved in the same hybridisation buffer. This equimolar mixture contained 50, 500, or 5,000 fmol of each of the following two species: (i) a Cy3-labelled oligonucleotide complementary to the probe on the microbeads (FB-Cy3-TargetA: 5′-CATTAGTTCC GGCCAGCAGA TTATAAATTT TTATGGTCCG TGGTTGTTCA CTCATTCTGA ATGCCATTAT GCAAGCCTCA CAATATAGTT AAATGCAATG-3′ (SEQ ID NO: 2); Integrated DNA Technologies, Inc.) and (ii) a Cy5-labelled oligonucleotide with a non-complementary sequence (FB-Cy5-TargetB: 5′-GAGTTGCCCA TCGATATGGG CAACTCTATC TGCACTGCTC ATTAATATAC TTCTGGGTTC CTTCCAGTTG TTTTTGCATA GTGATCAGCC TCTCTCTGAG-3′ (SEQ ID NO: 3); Integrated DNA Technologies, Inc.).
4. The pulse was pumped through the microbead bed and the fluorescence of the stream flowing out of the microbead bed was measured with a 474 HPLC scanning fluorescence detector (Waters Corp.). The fluorescence signal from each of the two oligonucleotides permitted the flow-through of each species to be determined.
5. The temperature of the capillary was reduced to 25° C. and the mobile phase was switched to a wash buffer containing 0.1×SSPE (Sigma-Aldrich Co. LLC) and 0.005% (v/v)N-lauroylsarcosine (Sigma-Aldrich Co. LLC), pumped at 50 μl/minute. This washing phase helped to remove non-hybridised oligonucleotides.
6. After washing was complete, the flow rate of the wash buffer was returned to 10 μl/minute and the temperature of the capillary was raised to 65° C. to release the hybridised oligonucleotides from the surface of the microbeads. The fluorescence signal from each of the two oligonucleotide species permitted their specific capture to be quantified.

### Results

A single 120-mer oligonucleotide was conjugated to an aliquot of microbeads via NHS/amine chemistry. The microbeads were packed into a short capillary terminated by a porous frit with a pore size smaller than the microbeads, trapping them inside the capillary. Hybridisation buffer was pumped through the capillary to block the microbeads.

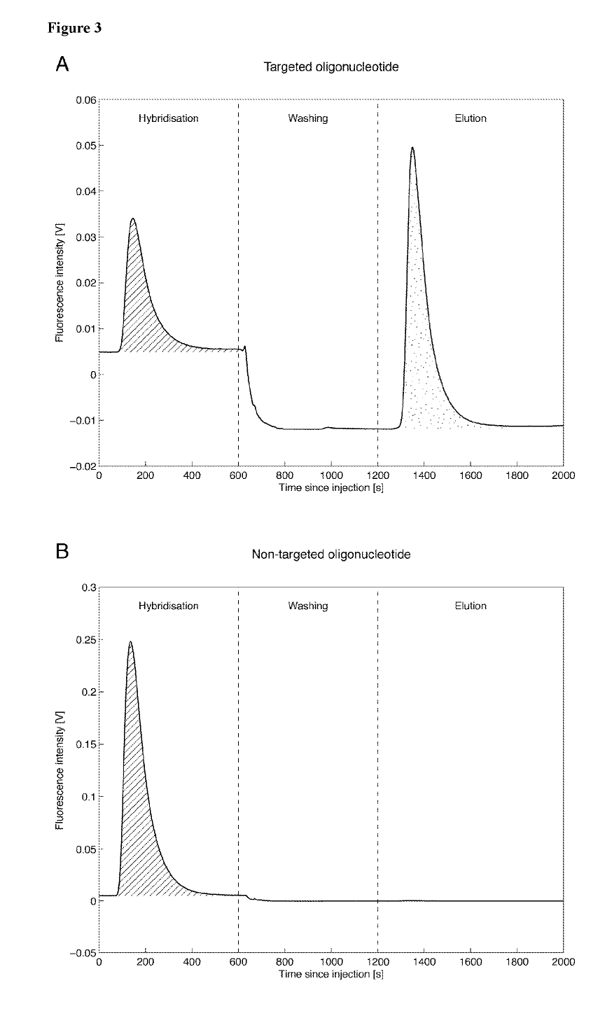

An HPLC injection valve was used to introduce a pulse of two fluorescently-labelled oligonucleotides into a stream of hybridisation buffer. The outflow from the bed was monitored in the green and red fluorescence channels using an HPLC fluorescence detector (FIG. 3). One oligonucleotide, labelled with the green dye Cy3, was complementary to the probe oligonucleotide on the microbeads and the majority of its molecules hybridised. Some molecules were not captured and passed straight through the bed, as illustrated by the peak in outflow fluorescence between 0 and 600 seconds (striped area). The mobile phase was then switched to a cold, but stringent, washing buffer during the ‘washing’ phase. To determine how much of the labelled oligonucleotide had actually bound, the temperature was increased and the hybridised molecules melted off the probes and flowed out of the bed, as illustrated by the peak in fluorescence occurring between 1200 to 2000 seconds (stippled area). The relative areas of the two peaks indicate the fraction of targeted oligonucleotide molecules that were captured by the microbead surfaces. The other oligonucleotide, labelled with the red dye Cy5, was not complementary to the probe and did not hybridise: the vast majority of DNA flowed straight through during the ‘hybridisation’ step, leaving very little bound to the surface.

By varying the density of hybridisation probes on the microbeads and the concentration of oligonucleotides in the injected pulse it was possible to investigate the impact of each of these variables on capture efficiency (Table 1).

TABLE 1
Efficiency of capture of targeted and non-targeted oligonucleotides
at different probe densities and oligonucleotide concentrations.
Values that were not measured are indicated by ‘NM’.

| | Moles of | | | |
|---|---|---|---|---|
| Moles of | each injected | Cy3 oligo. | Cy5 oligo. | Enrichment |
| probe (pmol) | oligo. (fmol) | captured (%) | captured (%) | (fold) |
| 5 | 50 | 31.9 | NM | — |
| 5 | 500 | 11.8 | NM | — |
| 5 | 5000 | 4.60 | NM | — |
| 50 | 50 | 89.4 | NM | — |
| 50 | 500 | 64.5 | 0.440 | 147 |
| 50 | 5000 | 9.42 | NM | — |

The results indicate that the amount of probe on the microbeads and the amount of injected oligonucleotide both affected the overall capture of the targeted oligonucleotide. Capture efficiency was highest (89.4%) when the amount of probe was greatest (50 pmol) and the amount of injected oligonucleotide was lowest (50 fmol). Decreasing the amount of capture probe or increasing the amount of injected oligonucleotide had the effect of decreasing the capture of the targeted oligonucleotide. In the case where the capture of non-targeted oligonucleotide was also measured, the value determined was much lower than that for the targeted oligonucleotide (0.44 versus 64.5%). The ratio of these capture efficiencies indicates an overall enrichment factor of 147-fold was achieved. In all cases the hybridisation step lasted only 10 minutes.

### Example 2: Rapid Enrichment of a Single Target in the Lambda Phage Genome

To demonstrate and quantify the enrichment of a specific sequence from the complete genome of an organism, a library of fragments were generated from lambda phage genomic DNA and driven through a bed of microbeads functionalised with a probe complementary to one 120 bp target.

### Preparation of Probe-Functionalised Microbeads

A 120-mer oligonucleotide with a 5′ amine modification (FB-AmC6-ProbeA120: 5′-CCGTCAAAAA CATTGCATTT AACTATATTG TGAGGCTTGC ATAATGGCAT TCAGAATGAG TGAACAACCA CGGACCATAA AAATTTATAA TCTGCTGGCC GGAACTAATG AATTTATTGG-3′ (SEQ ID NO: 4); Integrated DNA Technologies, Inc.) was coupled to microbeads at 5 pmol/mg density in the same way as described in Example 1. This probe was targeted against a single region in the J02459 lambda phage reference genome (GenBank): bases 21930-22049.

### Preparation of Adapter Duplexes

1. A pair of complementary oligonucleotides were synthesised by Integrated DNA Technologies, Inc. The ‘top’ oligonucleotide was synthesised with a 5′ C3 cap and a phosphorothioate bond on the 3′ terminal base (FB-PreLig-Ad-5′-T: 5′-ACTCTTTCCC TACACGACGC TCTTCCGATC T-3′ (SEQ ID NO: 5)). The ‘bottom’ oligonucleotide was synthesised with a 5′ phosphate and 3′ C3 cap (FB-PreLig-Ad-5′-B: 5′-GATCGGAAGA GCGTCGTGTA GGGAAAGAGT-3′ (SEQ ID NO: 6)).
2. The oligonucleotides were resuspended at 30 μM concentration in a 50 μl volume of annealing buffer, containing 50 mM sodium chloride, 1 mM Tris-HCl, pH 7.5, and 100 μM EDTA (all from Sigma-Aldrich Co. LLC).
3. The mixture was denatured at 95° C. for 2 minutes, cooled slowly to 25° C. (0.1° C./second), maintained at 25° C. for 5 minutes, and then stored at 4° C. The pair of oligonucleotides annealed, creating the FB-PreLig-Ad-5′ adapter duplex.
4. The above steps were repeated with another pair of oligonucleotides to create the FB-PreLig-Ad-3′ adapter duplex. The ‘top’ oligonucleotide was synthesised with a 5′ phosphate and 3′ C3 cap (FB-PreLig-Ad-3′-T: 5′-GATCGGAAGA GCACACGTCT GAACTCCA-3′ (SEQ ID NO: 7)). The ‘bottom’ oligonucleotide was synthesised with a 5′ C3 cap and a phosphorothioate bond on the 3′ terminal base (FB-PreLig-Ad-3′-B: 5′-ACTCTTTCCC TACACGACGC TCTTCCGATC T-3′ (SEQ ID NO: 5)).

### Preparation of DNA for Enrichment

1. 2 μg of lambda phage genomic DNA (Promega Corp.) was randomly sheared into 180 bp double-stranded DNA fragments with an S2 focused ultrasonicator (Covaris Inc.), according to the manufacturer's instructions.
2. The sheared DNA was cleaned and size-selected with a 1× ratio of Agencourt AMPure XP beads (Beckman Coulter, Inc.), following the manufacturer's instructions. The elution volume was 27 μl of nuclease-free water.
3. 25 μl of the clean, sheared DNA was end-repaired and A-tailed by diluting it to 50 μl with the following components: 1×T4 DNA ligase buffer (Life Technologies), 1 mM dATP (Enzymatics, Inc.), 200 μM dNTP mixture (Enzymatics, Inc.), 100 U/ml Klenow fragment (Enzymatics, Inc.), 30 U/ml T4 DNA polymerase (Enzymatics, Inc.), 220 U/ml T4 polynucleotide kinase (Enzymatics, Inc.), and 0.3 U/μl Top DNA polymerase (Bioneer Corp.).
4. The sample was incubated at 25° C. for 30 minutes, heated to 72° C. for 30 minutes, and then cooled to 4° C.
5. 15 μl of reagents were added to the DNA sample to yield a 65 μl reaction with added ligation components: 2.3 μM FB-PreLig-Ad-5′ adapter duplex, 2.3 μM FB-PreLig-Ad-3′ adapter duplex, and 18,500 U/ml T4 DNA ligase (Life Technologies).
6. The sample was incubated at 20° C. for 15 minutes.
7. The ligated DNA was cleaned and size-selected with a 1.8× ratio of Agencourt AMPure XP beads, following the manufacturer's instructions. The elution volume was 30 μl of nuclease-free water.
8. The concentration of DNA in the clean, ligated DNA sample was determined using a Qubit dsDNA HS assay (Life Technologies).
9. The aliquoted DNA was amplified in a 50 μl ‘pre-capture’ PCR containing the following: 300 ng of clean, ligated DNA, 1× AccuBuffer (Bioline), 1 mM dNTP mixture (Bioline), 0.1/μl ACCUZYME DNA polymerase (Bioline), 1 μM forward PCR primer (FB-PCR1-5′: ACTCTTTCCC TACACGACGC TCTTCCGATC T (SEQ ID NO: 5); Integrated DNA Technologies, Inc.), and 1 μM reverse PCR primer (FB-PCR1-3′: TGGAGTTCAG ACGTGTGCTC TTCCGATCT (SEQ ID NO: 8); Integrated DNA Technologies, Inc.).
10. The sample was thermal-cycled as follows: a hot start of 98° C. for 3 minutes; 6 cycles of 98° C. for 30 seconds, 65° C. for 30 seconds, and 72° C. for 1 minute; a final extension step of 72° C. for 10 minutes; and cooled to 4° C.
11. The concentration of DNA in the clean, amplified DNA sample was determined using a Qubit dsDNA HS assay (Life Technologies).

### Enrichment

1. A hollow cylinder was milled from PTFE with a diameter of 10 mm and a length of 31 mm. An internal well with a diameter of 6 mm and a length of 10.7 mm tapered to a narrow slot with a width of about 1 mm and a length of about 10 mm. The bottom of the slot was sealed with a 0.5 μm-pore PEEK frit-in-a-ferrule (IDEX Corp.), which was screwed in from the other side of the PTFE column. A 100 μm-bore, 50 cm length of FEP capillary tubing (IDEX Corp.) connected this ferrule to a 1.5 ml microcentrifuge tube to permit collection of the outflow.
2. The column assembly was fitted into a custom rig milled from aluminium and attached to a CP-200HT-TT Peltier-based temperature controller (TE Technology, Inc.), enabling precise control of the temperature. A lid, also milled from aluminium, could be screwed onto the rig to allow the contents of the column to be pressurised with a source of compressed gas.
3. 250 μl of wash buffer containing 0.1×SSPE and 0.005% (v/v)N-lauroylsarcosine was pipetted into the well in the column.
4. The lid was closed and the headspace above the column was pressurised with nitrogen gas, pushing the buffer through the well, slot, frit, and capillary tubing into the microcentrifuge tube. A flow rate of about 5 μl/minute was maintained for 2.5 minutes to fully prime the flow path.
5. 25 μl of the previously prepared microbead suspension, equivalent to 0.5 mg, were resuspended in 10 μl of wash buffer and pipetted into the slot in the column assembly, beneath the surface of the wash buffer in the well.
6. The headspace was re-pressurised to drive the microbead suspension at a rate of about 5 μl/minute for 2.5 minutes. The frit at the base of the slot trapped the microbeads, causing them to pack into bed with a diameter of 1 mm and a depth of about 0.7 mm.
7. Wash buffer remaining in the well of the column assembly was discarded and replaced with 250 μl of hybridisation buffer consisting of 1×Hi-RPM buffer (Agilent Technologies, Inc.) and 50 ng/μl salmon sperm DNA (Life Technologies).
8. The column assembly was heated to 65° C. with the Peltier system and the headspace above was pressurised to drive the hybridisation buffer through the microbead bed at a rate of about 5 μl/minute. This flow was maintained for 10 minutes to pre-hybridise (block) the microbeads and other surfaces inside the column assembly.
9. 300 ng of the clean, amplified DNA sample were dissolved in 8 μl hybridisation buffer supplemented with 3.125% (v/v) glycerol (Sigma-Aldrich Co. LLC) and 10 μM of an oligonucleotide to block the 5′ adapter (FB-PreLig-Block-5′: 5′-ACTCTTTCCC TACACGACGC TCTTCCGATC T-3′ (SEQ ID NO: 5); Integrated DNA Technologies, Inc.) and an oligonucleotide to block the 3′ adapter (FB-PreLig-Block-3′: 5′-TGGAGTTCAG ACGTGTGCTC TTCCGATCT-3′ (SEQ ID NO: 8); Integrated DNA Technologies, Inc.).
10. The temperature of the rig was decreased to 25° C.
11. This DNA sample was denatured at 98° C. for 5 minutes, cooled to 65° C. for 5 minutes, and then stored at 4° C. for 5 minutes. The 65° C. step permitted the blocking oligonucleotides to hybridise to the adapter sequences in the DNA sample, preventing the formation of ‘daisy-chains’ that could impede enrichment.
12. 2 μl of the denatured DNA sample, containing 75 ng of DNA, were pipetted into the slot of the column assembly using a pipette. The glycerol in the sample caused it to sink through the hybridisation buffer and collect above the bed of microbeads.
13. The temperature of the rig was increased to 65° C. and the headspace was repressurised, forcing the DNA sample through the packed bed of microbeads at a flow rate of about 5 μl/minute for 10 minutes.
14. The temperature of the rig was decreased to 25° C.
15. The buffer in the well of the column assembly was replaced with 250 μl of wash buffer and the headspace was repressurised to drive the buffer through the microbeads at about 30 μl/minute for 5 minutes.
16. The previous step was repeated 4 more times with fresh wash buffer.
17. Fresh wash buffer was pipetted into the column and driven through the microbead bed at about 10 μl/minute. As the buffer flowed, the temperature of the column assembly was raised to 95° C. to release the hybridised DNA from the surface of the microbeads. During this time, the outflow from the column assembly was collected in a clean 1.5 ml microcentrifuge tube.

### Recovery and Sequencing of Enriched DNA

1. The enriched DNA was amplified in a 50 μl ‘post-capture’ PCR containing the following: 14 μl eluted DNA, 1×AccuBuffer (Bioline), 1 mM dNTP mixture (Bioline), 0.1/μl ACCUZYME DNA polymerase (Bioline), 200 nM forward PCR primer (FB-PCR2-5′: 5′-AATGATACGG CGACCACCGA GATCTACACT CTTTCCCTAC ACGACGCTCT TCCGATC-3′ (SEQ ID NO: 9); Integrated DNA Technologies, Inc.), and 200 nM reverse PCR primer (FB-PCR2-3′-Index39: 5′-CAAGCAGAAG ACGGCATACG AGATGCACTT GTGACTGGAG TTCAGACGTG TGCTC-3′ (SEQ ID NO: 10); Integrated DNA Technologies, Inc.).
2. The sample was thermal-cycled as follows: a hot start of 98° C. for 3 minutes; 16 cycles of 98° C. for 30 seconds, 65° C. for 30 seconds, and 72° C. for 1 minute; a final extension step of 72° C. for 10 minutes; and cooled to 4° C.
3. The amplified DNA was cleaned and size-selected with a 1.8×ratio of Agencourt AMPure XP beads, following the manufacturer's instructions. The elution volume was 30 μl of nuclease-free water.
4. The clean, amplified DNA was pooled with other samples and sequenced using a MiSeq Desktop Sequencer (Illumina Corp.) and a 600-cycle MiSeq Reagent Kit v3 (Illumina Corp.), following the manufacturer's instructions.
5. Reads were trimmed to 100 bases and aligned to the J02459 lambda phage reference genome using a bioinformatics pipeline based on: BWA version 0.6.2 [23], FastQC version 0.10.1 (Babraham Institute), Genome Analysis Toolkit version 1.6-13 [24], Picard version 1.104, and SAMtools version 0.1.18 [25].

### Results

A single 120-mer oligonucleotide, complementary to one region of the lambda genome, was conjugated to an aliquot of microbeads via NHS/amine chemistry. These microbeads were packed into a PTFE column which integrated a porous frit with a pore size smaller than the microbeads. Hybridisation buffer was pumped through the microbead bed to block subsequent non-specific interactions.

Lambda phage genomic DNA was fragmented by sonication, end-repaired, and A-tailed. Adapter duplexes with T-overhangs were prepared by annealing complementary oligonucleotides and then ligated to the repaired fragments. These ligated fragments were then amplified by PCR and resuspended in hybridisation buffer. This sample was then pipetted onto the microbead bed and the flow was restarted, forcing the DNA between the microbeads and facilitating hybridisation of the targeted fragments. After hybridisation, non-targeted DNA was washed away by pumping wash buffer through the packed bed. DNA hybridised to the microbeads was released by raising the temperature. The outflow from the column during this elution step was collected, amplified by PCR, and then sequenced by NGS.

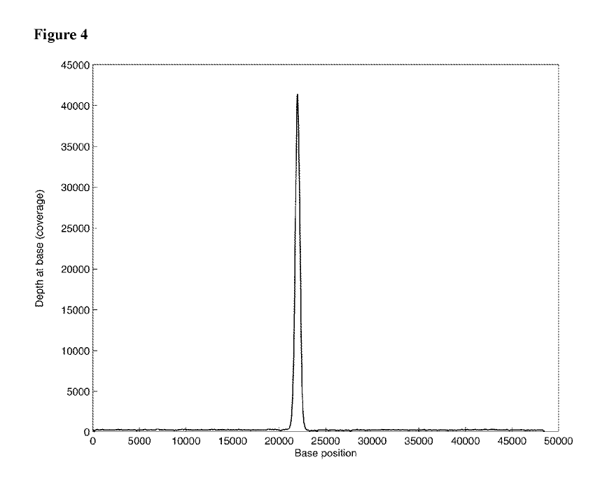

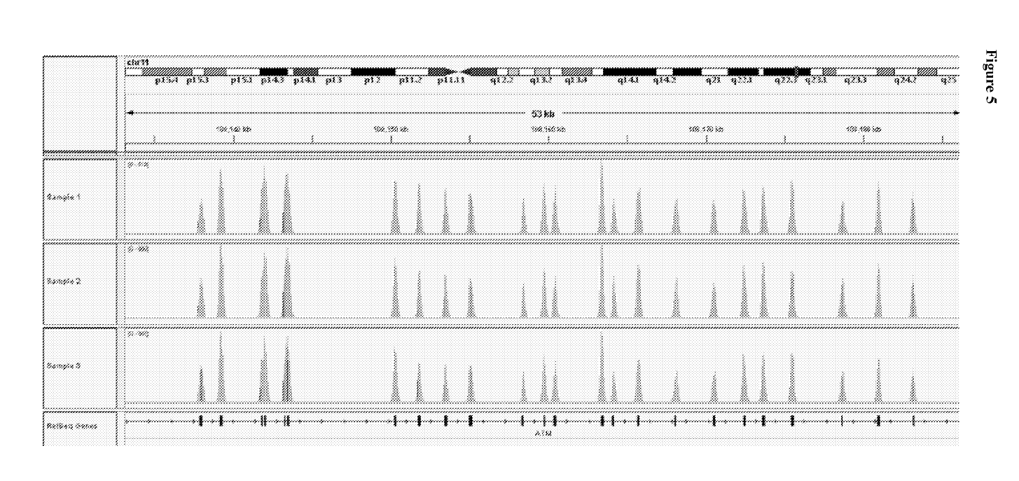

486,000 paired-end reads were aligned to the lambda reference genome and used to calculate enrichment metrics. As shown in FIG. 4, plotting the number of reads against every base in the lambda genome revealed a spike corresponding to the 120 bp targeted region (GenBank J02459, 21930-22049). The average number of reads in this region +/−250 bp flanks was 32,294 reads, versus 329 for the rest of the genome. Consequently, the enrichment of the baited region was 98-fold, achieved with a 10-minute hybridisation step.

### Example 3: Enrichment of Multiple Genes in the Human Genome

### Preparation of Probe-Functionalised Microbeads

Microbeads were prepared in the same way as described in Example 1 with the following modification: 876 120-mer oligonucleotide probes were coupled to the microbeads at a density of 0.5 pmol/mg each. These probes were designed against 135 regions in 5 genes of the GRCh37 human reference genome (Genome Reference Consortium): ATM (chr11:108098327-108236260), BRCA1 (chr17:41197670-41276138), BRCA2 (chr13:32890573-32972932), PALB2 (chr16:23614755-23652503), and TP53 (chr17:7572902-7579937). The probes covered all exons, with 25 bp flanks, and tiled to a depth of 3×. The total targeted territory was 36.8 kb and the total baited territory was 45.5 kb.

### Preparation of DNA for Enrichment

DNA was prepared in the same way as described in Example 2 with the following modification: the genomic DNA was human (Promega Corp.).

### Enrichment

1. An Oligo Clean & Concentrator column (Zymo Research Corp.) was fitted into a custom rig milled from aluminium and attached to a CP-200HT-TT Peltier-based temperature controller (TE Technology, Inc.), enabling precise control of the temperature. A lid, also milled from aluminium, could be screwed onto the rig to allow the contents of the column to be pressurised with a source of compressed gas.
2. 50 μl of the previously prepared microbead suspension, equivalent to 1 mg, was pipetted into the Zymo column.
3. The lid was closed and the headspace above the Zymo column was pressurised to 50 mbar, pushing the suspension through the column. The frit in the Zymo column caused the microbeads to collect into a bed with a diameter of about 2 mm and a depth of about 0.35 mm. The outflow of the column dripped into a 1.5 microcentrifuge tube positioned underneath. Pressure was maintained for 2.5 minutes, forcing the entire volume of buffer out of the column, but leaving the microbead bed ‘damp’.
4. A blocking buffer was prepared, containing 5.38 mM EDTA (Life Technologies), 750 ng/μl salmon sperm DNA (Life Technologies), 5.44×Denardt's solution (Life Technologies), 0.11% SDS (Life Technologies), 5.43×SSPE (Sigma-Aldrich Co. LLC), 10 μM FB-PreLig-Block-5′, and 10 μM FB-PreLig-Block-3′. 100 μl was pipetted onto the microbead bed.
5. The Zymo column was heated to 65° C. with the Peltier system and the headspace above was pressurised to 50 mbar for 2.5 minutes to drive the blocking buffer through the microbead bed at a rate of microliters/minute. This step pre-hybridised (blocked) the microbeads and other surfaces inside the Zymo column.
6. The temperature of the rig was decreased to 25° C.
7. A hybridisation buffer was prepared, containing the same components as the blocking buffer, but with 10-fold greater concentrations of the two blocking oligonucleotides (100 μM each). 750 ng of the previously prepared adapter-modified DNA sample were dissolved in 50 μl of this buffer.
8. This DNA sample was denatured at 98° C. for 5 minutes, cooled to 65° C. for 5 minutes, and then stored at 4° C. for 5 minutes.
9. 5 μl of the denatured DNA sample, containing 75 ng of DNA, were pipetted onto the microbead bed.
10. The temperature of the rig was increased to 65° C. and the headspace was repressurised to 50 mbar for 2.5 minutes, forcing the DNA sample through the packed bed of microbeads at a rate of microliters/minute.
11. The temperature of the rig was decreased to 55° C.
12. A wash buffer was prepared, containing 0.1% SDS and 0.1×SSC (Sigma-Aldrich Co. LLC).
13. 200 μl of the wash buffer, supplemented with 1 μM FB-PreLig-Block-5′, and 1 μM FB-PreLig-Block-3′, were pipetted onto the microbead bed. The headspace was repressurised to 50 mbar for 2.5 minutes to drive the buffer through the microbeads at a rate of microliters/minute.
14. The previous step was repeated 7 more times with 200 μl aliquots of wash buffer that were not supplemented with blocking oligonucleotides.
15. The temperature of the rig was decreased to 25° C.
16. Hybridised DNA was released from the microbeads by pipetting 100 μl of 100 mM NaOH (Sigma-Aldrich Co. LLC) onto the microbead bed and letting them incubate for 5 minutes.
17. The headspace was repressurised to 50 mbar for 5 minutes and the eluate was collected in a clean 1.5 ml microcentrifuge tube.
18. 100 μl of Tris-HCl, pH 7.5 (Sigma-Aldrich Co. LLC), was added to the eluted DNA to neutralise the pH.
19. The enriched DNA was cleaned using a DNA Clean & Concentrator-5 column (Zymo Research Corp.), following the manufacturer's instructions. The DNA Binding Buffer:DNA ratio used was 7 and the elution volume was 32 μl of nuclease-free water.

### Recovery and Sequencing of Enriched DNA

1. The enriched DNA was amplified in a 50 μl ‘post-capture’ PCR containing the following: 30 μl clean, enriched DNA, 1×AccuBuffer (Bioline), 1 mM dNTP mixture (Bioline), 0.1/μl ACCUZYME DNA polymerase (Bioline), 200 nM forward PCR primer (FB-PCR2-5′: 5′-AATGATACGG CGACCACCGA GATCTACACT CTTTCCCTAC ACGACGCTCT TCCGATC-3′ (SEQ ID NO: 9); Integrated DNA Technologies, Inc.), and 200 nM reverse PCR primer (FB-PCR2-3′-Index6: 5′-CAAGCAGAAG ACGGCATACG AGATGCCAAT GTGACTGGAG TTCAGACGTG TGCTC-3′ (SEQ ID NO: 10); Integrated DNA Technologies, Inc.).
2. The sample was thermal-cycled as follows: a hot start of 98° C. for 3 minutes; 16 cycles of 98° C. for 30 seconds, 65° C. for 30 seconds, and 72° C. for 1 minute; a final extension step of 72° C. for 10 minutes; and cooled to 4° C.
3. The amplified DNA was cleaned using a DNA Clean & Concentrator-5 column (Zymo Research Corp.), following the manufacturer's instructions. The DNA Binding Buffer:DNA ratio used was 5 and the elution volume was 30 μl of nuclease-free water.
4. The clean, amplified DNA was pooled with other samples and sequenced using a MiSeq Desktop Sequencer (Illumina Corp.) and a 150-cycle MiSeq Reagent Kit v3 (Illumina Corp.), following the manufacturer's instructions.
5. 75-base reads were aligned to the GRCh37 human reference genome using a bioinformatics pipeline based on: BWA version 0.6.2 [23], dsSNP build 135 [26], FastQC version 0.10.1 (Babraham Institute), Genome Analysis Toolkit version 1.6-13 [24], Picard version 1.107, and SAMtools version 0.1.18 [25], VCFtools version 0.1 [27], VEP script [28].

### Results

Human genomic DNA was fragmented by sonication, end-repaired, and A-tailed. Adapter duplexes were ligated to these fragments, which were then amplified by PCR and resuspended in hybridisation buffer. This sample was then pumped through a bed of microbeads packed into a disposable commercial DNA clean-up column, which incorporates a frit. The microbeads were conjugated with 876 oligonucleotide probes against five genes in the human genome: ATM, BRCA1, BRCA2, PALB2, and TP53. After hybridisation, non-targeted DNA was washed away by pumping wash buffer through the packed bed. Targeted DNA, still hybridised to the microbeads, was released by raising the pH with sodium hydroxide. The released DNA was then collected from the column, amplified by PCR, and then sequenced by NGS.

3,030,000 paired-end reads were aligned to the human genome and used to calculate enrichment metrics. Mean coverage in the targeted regions was 236 unique reads with 100% of the targeted bases having at least 50 reads. The percentage ‘selected bases’, i.e. base reads within the baited and near-baited (+/−250 bp) regions, was 7.16%. Consequently, the enrichment of the baited region was 4000-fold, achieved with a 2.5-minute hybridisation step.

### Example 4: Enrichment of Multiple Genes in the Human Genome Using Two Hybridisation Steps

### Preparation of Probe-Functionalised Microbeads

Microbeads were prepared in the same way as described in Example 3.

### Preparation of DNA for Enrichment

DNA was prepared in the same way as described in Example 3.

### Hybridisation 1

### Preparation and Loading of the Microbeads

1. A custom designed hybridisation column (comprising a porous, hydrophilic/polyethylene frit and a pore size 7-12 μm with a column thickness of 2.0 mm) was fitted into a custom rig and attached to a Peltier-based temperature controller enabling precise control of the temperature. A lid could be screwed onto the rig to allow the contents of the column to be pressurised with a source of compressed gas.
2. 50 μl of the previously prepared microbead suspension (20 mg/ml), was pipetted into the centre of each hybridisation column, just above the frit.
3. The lid was closed and the headspace above the column was pressurised to 50 mbar, pushing the suspension through the column. The outflow of the column dripped into a microcentrifuge tube or a 96-well plate which was positioned underneath. Pressure was maintained for 2.5 minutes, forcing the entire volume of buffer out of the column, but leaving the microbead bed ‘damp’.

### Pre-Hybridisation Phase

1. A blocking buffer was prepared, containing 5.38 mM EDTA (Life Technologies), 750 ng/μl salmon sperm DNA (Life Technologies), 5.44×Denardt's solution (Life Technologies), 0.11% SDS (Life Technologies), 5.43×SSPE (Sigma-Aldrich Co. LLC), 10 μM FB-PreLig-Block-5′, and 10 μM FB-PreLig-Block-3′. 100 μl was pipetted onto the microbead bed.
2. The column was heated to 65° C. with the Peltier system and the headspace above was pressurised to 50 mbar for 2.5 minutes to drive the blocking buffer through the microbead bed at a rate of microliters/minute. This step pre-hybridised (blocked) the microbeads and other surfaces inside the column.
3. The outflow of the column dripped into a microcentrifuge tube positioned underneath. Pressure was maintained for 2.5 minutes, forcing the entire volume of buffer out of the column, but leaving the microbead bed ‘damp’.
4. The temperature of the rig was maintained at 65° C. for the next phase of loading the DNA sample for the first hybridisation step.

### Denaturing the DNA

1. A hybridisation buffer was prepared, containing the same components as the blocking buffer, but with 10-fold greater concentrations of the two blocking oligonucleotides (100 μM each).
2. 300 ng of the previously prepared adapter-modified DNA sample was dried down and added to 5 μl of the hybridisation buffer, then briefly vortex and centrifuged.
3. This DNA sample was denatured at 98° C. for 5 minutes, cooled to 65° C. for 5 minutes, and then stored at 4° C. for 5 minutes.

### Hybridisation Phase

1. 5 μl of the denatured DNA sample, containing 300 ng of DNA, was pipetted onto the microbead bed.
2. With the temperature of the rig still at 65° C., the headspace was repressurised to 50 mbar for 2.5 minutes, forcing the DNA sample through the packed bed of microbeads.

### Rinse and Wash Phase

1. The temperature of the rig was decreased to 55° C.
2. A wash buffer was prepared, containing 0.1% SDS and 0.1×SSC (Sigma-Aldrich Co. LLC).
3. 200 μl of rinsing buffer (the wash buffer, supplemented with 1 μM FB-PreLig-Block-5′, and 1 μM FB-PreLig-Block-3′) was pipetted onto the microbead bed. The headspace was repressurised to 100 mbar for 3.5 minutes to drive the rinsing buffer through the microbeads.
4. The previous step was repeated 5 more times with 200 μl aliquots of wash buffer that were not supplemented with blocking oligonucleotides.

### Elution Phase

5. The temperature of the rig was decreased to 25° C.
6. Hybridised DNA was released from the microbeads by pipetting 100 μl of 100 mM NaOH (Sigma-Aldrich Co. LLC) onto the microbead bed and letting them incubate for 5 minutes.
7. The headspace was repressurised to 50 mbar for 5 minutes. The eluate was collected in a microcentrifuge tube or a 96-well plate block.

### Neutralisation and Clean-Up of the Eluted DNA

8. 100 μl of Tris-HCl, pH 7.5 (Sigma-Aldrich Co. LLC), was added to the eluted DNA to neutralise the pH.
9. The enriched DNA was cleaned using a DNA Clean & Concentrator-5 column (Zymo Research Corp.), following the manufacturer's instructions. The DNA Binding Buffer:DNA ratio used was 7 and the elution volume was 32 μl of nuclease-free water.

### Post-Capture PCR

1. Each of the DNA samples was amplified in a 50 μl ‘post-capture’ PCR containing the following: 30 μl clean, enriched DNA, 1×AccuBuffer (Bioline), 1 mM dNTP mixture (Bioline), 0.1/μl ACCUZYME DNA polymerase (Bioline), 200 nM forward PCR primer (FB-PCR2-5′: 5′-AATGATACGG CGACCACCGA GATCTACACT CTTTCCCTAC ACGACGCTCT TCCGATC-3′ (SEQ ID NO: 9); Integrated DNA Technologies, Inc.) and 200 nM reverse PCR primer (FB-PCR2-3′-Index1: 5′-CAAGCAGAAGA CGGCATACGA GATCGTGATG TGACTGGAGT TCAGACGTGT GCTC-3′ (SEQ ID NO: 11)).
2. The sample was thermal-cycled as follows: a hot start of 98° C. for 3 minutes; 20 cycles of 98° C. for 30 seconds, 65° C. for 30 seconds, and 72° C. for 1 minute; a final extension step of 72° C. for 10 minutes; and cooled to 4° C.
3. The amplified DNA was cleaned using a DNA Clean & Concentrator-5 column (Zymo Research Corp.), following the manufacturer's instructions. The DNA Binding Buffer:DNA ratio used was 5 and the elution volume was 30 μl of nuclease-free water.
4. The concentration of DNA in the clean, ligated DNA sample was determined using a Qubit dsDNA HS assay (Life Technologies).
5. A High-Sensitivity D1K ScreenTape was used to determine the peak fragment size which should be between 270 and 320 bp.

### Hybridisation 2

The enrichment process of hybridisation 1 was repeated using 300 ng of the amplified, cleaned and enriched DNA recovered from hybridisation 1. The recovered sample was subjected to a further round of post-capture PCR.

### Sequencing of Enriched DNA

The clean, amplified and enriched DNA recovered from hybridisation 2 was pooled and sequenced using a MiSeq Desktop Sequencer (Illumina Corp.) and a 150-cycle MiSeq Reagent Kit v3 (Illumina Corp.), following the manufacturer's instructions.

### Results

Human genomic DNA was fragmented by sonication, end-repaired and A-tailed. Adapter duplexes were ligated to these fragments, which were then amplified by PCR and resuspended in hybridisation buffer. The samples were then pushed under pressure through a bed of microbeads compressed on top of a frit. Custom made columns were used which incorporate a Porex frit (Frit Make: Porex, Pore size 7-12 μm, Thickness—2.0 mm, Material—XS-82591). The custom made columns replaced the previously used disposable commercial DNA clean-up columns. The microbeads were conjugated with 876 oligonucleotide probes against five genes in the human genome: ATM, BRCA1, BRCA2, PALB2 and TP53. Each hybridisation was only 150 sec long. After hybridisation, non-targeted DNA was washed away by pushing wash buffer through the packed bead bed under pressure. Targeted DNA, still hybridised to the microbeads, was released by raising the pH with sodium hydroxide. The released DNA was then collected from the column, cleaned and amplified by PCR.

Two rounds of hybridisation were used. In the first round of hybridisation, the targeted DNA was hybridised to the microbeads and the non-targeted DNA was washed away. The targeted DNA was released by raising the pH, collected from the column and amplified by PCR. In the second round of hybridisation the targeted DNA was the amplified product from hybridisation 1 and the protocol followed the same steps as above.

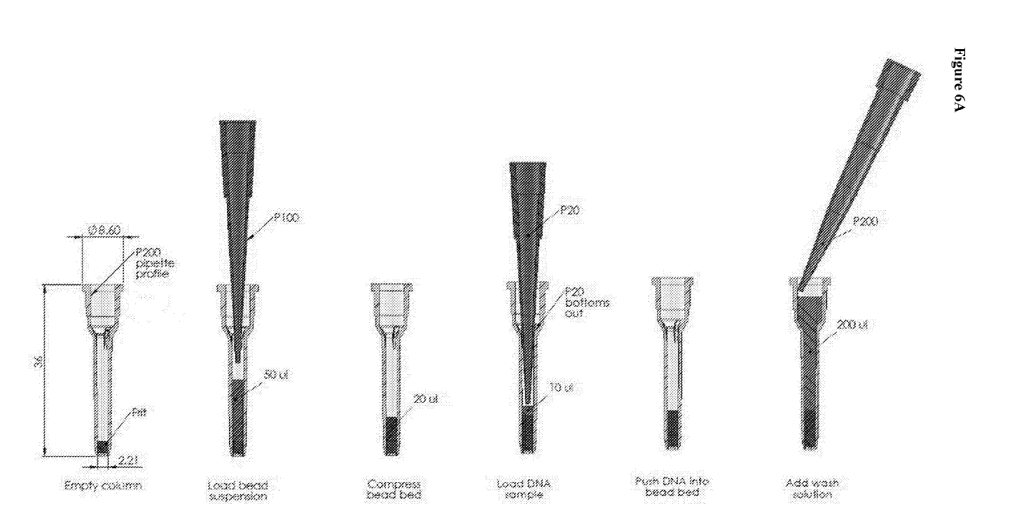

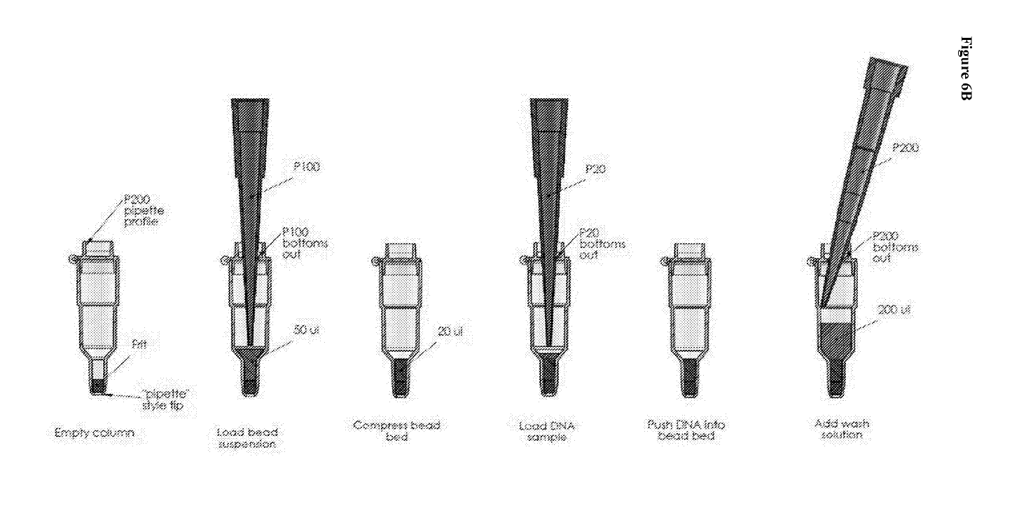

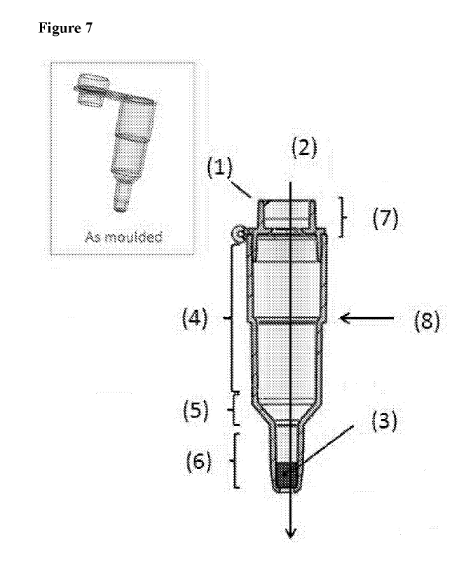

An initial data set was generated with HapMap DNA samples (http://hapmap.ncbi.nlm.nih.gov/hapmappopulations.html). All samples met the clinical metrics for the tested 5-gene panel (48 samples/MiSeq run, target coverage of >99% bases at a depth ≧30). Repeating the hybridisation step resulted in a >14,000-fold enrichment of the baited regions, i.e. a more than 3-fold improvement over the single hybridisation step used in Example 3. Enrichment of the target regions was specific, reproducible and uniform (see FIG. 5, which shows the results for the ATM gene). Mean target coverage was >600. 100.0% of the targeted bases were covered at >30× depth. The data are summarised in Table 2.

TABLE 2

| | | | | | % Target | | |
|---|---|---|---|---|---|---|---|
| | | Selected | Mean | | Bases with | | |
| | Total | Bases | Target | Fold | Depth ≧ | | Duplication |
| Sample # | Reads | (%) | Coverage | Enrichment | 30x | 50x | (%) |
| 1 | 1,940,644 | 26 | 616 | 15230 | 100.00 | 99.99 | 13.4 |
| 2 | 1,654,316 | 29 | 573 | 16803 | 100.00 | 99.98 | 14.4 |
| 3 | 1,799,918 | 30 | 651 | 17483 | 100.00 | 100.00 | 14.0 |
| 4 | 1,654,248 | 28 | 563 | 16201 | 100.00 | 100.00 | 13.0 |
| 5 | 1,840,250 | 31 | 738 | 18184 | 100.00 | 100.00 | 9.8 |
| 6 | 1,577,008 | 29 | 558 | 17240 | 100.00 | 100.00 | 15.3 |
| 7 | 1,916,692 | 25 | 635 | 14815 | 100.00 | 100.00 | 7.7 |
| 8 | 1,618,434 | 38 | 740 | 22620 | 100.00 | 100.00 | 17.6 |

### Example 5: Hereditary/Germline Variant Detection from Whole Blood DNA

### Preparation of Probe-Functionalised Microbeads

Microbeads were prepared in the same way as described in Example 3.

### Preparation of DNA for Enrichment

DNA was prepared in the same way as described in Example 3 with the following modification: the genomic DNA was extracted from whole blood.

### Enrichment of DNA

DNA was enriched in the same was described in Example 4.

### Results

The experiment of Example 4 using HapMap DNA samples was repeated with clinical DNA samples. 48 samples were run per MiSeq lane. The data from this experiment are summarised in Table 3.

Using the set-up described in Example 4, germline/hereditary variants of >50% frequency could be detected in DNA isolated from standard whole-blood DNA samples, therefore demonstrating that microbead-based hybridisation and enrichment produce sufficient sequencing depth to reliably detect germline/hereditary variants in high-quality, genomic DNA samples.

TABLE 3

| | | | | | % Target | | |
|---|---|---|---|---|---|---|---|
| | | Selected | Mean | | Bases with | | |
| | Total | Bases | Target | Fold | Depth ≧ | | Duplication |
| Sample # | Reads | (%) | Coverage | Enrichment | 30x | 50x | (%) |
| 1 | 1,363,484 | 31 | 382.74 | 17748 | 99.87 | 99.85 | 33.40 |
| 2 | 1,563,518 | 24 | 399.94 | 13010 | 99.92 | 99.88 | 17.90 |
| 3 | 1,246,672 | 28 | 391.10 | 15809 | 99.92 | 99.88 | 17.31 |
| 4 | 1,317,570 | 28 | 351.87 | 15187 | 99.92 | 99.87 | 25.61 |
| 5 | 1,360,830 | 26 | 394.17 | 14662 | 99.93 | 99.89 | 17.19 |
| 6 | 1,301,994 | 27 | 346.98 | 14944 | 99.91 | 99.88 | 25.33 |
| 7 | 1,418,390 | 24 | 338.49 | 13410 | 99.89 | 99.87 | 25.69 |
| 8 | 1,284,642 | 26 | 393.81 | 15234 | 99.98 | 99.92 | 17.06 |

The results were independently validated with clinical samples from 45 individuals testing a subset of 3 genes (TP53, BRCA1, BRCA2) using the Illumina TruSight Cancer panel or a Fluidigm PCR panel combined with Sanger sequencing. 44 out of 45 samples met the clinical required metric. One sample only just missed these metrics due to the low concentration of the supplied DNA. Mean target coverage was about 50% lower at >300. Fold enrichment was comparable at >12,000. The data are summarised in Table 4.

TABLE 4

| | | | | | % Target | | |
|---|---|---|---|---|---|---|---|
| | | Selected | Mean | | Bases with | | |
| | Total | Bases | Target | Fold | Depth ≧ | | Duplication |
| Metrics | Reads | (%) | Coverage | Enrichment | 30x | 50x | (%) |
| Min | 961,960 | 14 | 174 | 8,277 | 99.61 | 98.87 | 8.5 |
| Mean | 1,376,772 | 22 | 324 | 12,629 | 99.92 | 99.77 | 22.1 |
| Max | 1,916,802 | 31 | 457 | 17,748 | 100.00 | 100.00 | 41.6 |

A comparison of both data sets showed, that the set-up of Example 4—when applied to high-quality, blood-derived DNA samples—was able to detect 100% of the variants that had been identified with either the Illumina TruSight Cancer Panel or the Fluidigm PCR panel and validated with Sanger sequencing were detected: 461/461 total, which included 41 unique variants across all 45 clinical samples. For example, deletions such as 9 bp deletion in the TP53 gene (c.762_770delCATCACACT) were clearly identified in the tested clinical samples.

### Example 6: Somatic Variant Detection in DNA Derived from Whole Blood Preparations

### Preparation of Probe-Functionalised Microbeads

Microbeads were prepared in the same way as described in Example 3 using a panel with probes designed against 21 key genes known to contain driver mutations for a range of myeloproliferative neoplasms including polycythaemia vera (PV), essential thrombocythaemia (ET) and myelofibrosis (MF). The gene content targets ‘hot-spot’ exons where clinically relevant mutations are known and every exon for tumour suppressor, hereditary and highly implicated research-related genes. The genes included were ASXL-1, CBL, CALR, CKIT, CSF3R, EZH2, IDH1, IDH2, JAK2, MPL, NRAS, KRAS, RUNX1, SETBP1, SRSF2, TP53, TET2, DNMT3A, U2AF1, SF3B1, SH2B with a total target size of 37.6 kb.

### Preparation of DNA for Enrichment

DNA was prepared from HapMap DNA samples in the same way as described in Example 5.

### Enrichment of DNA

DNA was enriched in the same was described in Example 4

### Results

Using the set-up described in Example 4 and standard whole-blood as source of the DNA samples, the 21-gene panel described above was able to detect somatic variants of low frequency (>1%) observed in only a small percentage of reads at any locus. The results demonstrates that this set-up provides high-stringent sensitivity to detect low-frequency in good-quality DNA samples.

Enrichment resulted in a ˜10,000-fold enrichment of the baited regions, i.e. only 30% less than the enrichment of Example 4 using a germline DNA sample. Mean target coverage was ˜1000× (see Table 5). Table 6 shows the coverage across the sites of interest observed in 12 HapMap DNA samples.

The data confirm that the panel could reliably detect somatic mutations at the following sites of interest in MPNs: JAK2 (exon 12—AAs 536-547), JAK2 (V617F), MPL (W515), KIT (D816V), and TET2 (R550).

Using the same set-up, analysis of clinical research samples further showed that the panel was able to reliably detect not only SNVs but also deletions of up to 52 bp, which are particularly informative in the CALR gene.

TABLE 5

| | | | Mean | | Dupli- |
|---|---|---|---|---|---|
| Sample | | Selected | Target | Fold | cation |
| # | Total Reads | Bases (%) | Coverage | Enrichment | (%) |
| 1 | 4,408,302 | 25 | 1008 | 12078 | 20.5 |
| 2 | 4,982,940 | 15 | 826 | 7501 | 6.2 |
| 3 | 4,630,950 | 24 | 1062 | 11866 | 18.5 |
| 4 | 5,257,924 | 27 | 1372 | 13240 | 18.0 |
| 5 | 5,792,398 | 21 | 1149 | 10153 | 17.8 |
| 6 | 5,146,086 | 18 | 944 | 8771 | 11.9 |
| 7 | 5,199,820 | 13 | 772 | 6664 | 5.6 |
| 8 | 5,505,042 | 24 | 1182 | 11395 | 20.5 |
| 9 | 5,815,684 | 25 | 1423 | 12247 | 16.6 |
| 10 | 6,047,848 | 19 | 1189 | 9492 | 13.0 |
| 11 | 4,729,418 | 24 | 1149 | 11733 | 12.8 |
| 12 | 5,456,088 | 23 | 1294 | 11446 | 13.4 |

TABLE 6

| Name | S1 | S2 | S3 | S4 | S5 | S6 | S7 | S8 | S9 | S10 | S11 | S12 |
|---|---|---|---|---|---|---|---|---|---|---|---|---|
| MPL_W515 | 546 | 810 | 762 | 1031 | 964 | 732 | 700 | 982 | 1234 | 1055 | 746 | 987 |
| KIT_D816V | 1142 | 792 | 1075 | 1319 | 1121 | 898 | 721 | 1119 | 1375 | 1098 | 1197 | 1189 |
| TET2_R550 | 834 | 617 | 787 | 1116 | 879 | 809 | 649 | 819 | 1144 | 1026 | 930 | 939 |
| JAK2_Exon_12 | 892 | 647 | 833 | 1013 | 889 | 761 | 546 | 882 | 1001 | 823 | 918 | 800 |
| JAK2_V617F | 1083 | 810 | 1143 | 1334 | 1092 | 958 | 752 | 1215 | 1333 | 1158 | 1251 | 1188 |

### Example 7: Somatic Variant Detection in DNA Derived from FFPE Tissue

### Preparation of Probe-Functionalised Microbeads

Microbeads were prepared in the same way as described in Example 3.

### Preparation of DNA for Enrichment

DNA was prepared in the same way as described in Example 3 with the following modification: the genomic DNA was extracted from Formalin-Fixed Paraffin-Embedded (FFPE) cancer tissues.

### Enrichment of DNA

DNA was enriched in the same way as described in Example 4

### Results

Using a 5-gene panel and the set-up described in Example 4, somatic variants of low frequency (>1%) from DNA derived from FFPE tissue samples could also be detected. FFPE samples are a common source of biological material for solid cancer diagnosis and scientific research, but they can be difficult to work with because of the poor quality of extracted DNA as a result of the preparation and/or fixation process which leads to severe degradation, damage and molecular or biological modification of the DNA. As a consequence, FFPE samples often yield only low quantities of usable DNA.

Despite these challenges, the set-up described in Example 4 resulted in a 6500 fold enrichment of target DNA from FFPE breast cancer tissue (see Table 7), i.e. only 50% less than the enrichment of Example 4, which used a high-quality DNA sample, and only 35% less than the enrichment of Example 5, which used whole-blood clinical DNA samples. At 24 samples per MiSeq lane, mean target coverage was ˜300-500. Coverage could be doubled if only 12 samples are run per MiSeq lane, resulting in 600-1000 target coverage which is more suitable for somatic variant calling.

TABLE 7

| | | | Mean | | Dupli- |
|---|---|---|---|---|---|
| Sample | | Selected | Target | Fold | cation |
| # | Total Reads | Bases (%) | Coverage | Enrichment | (%) |
| 1 | 3,048,166 | 11 | 347 | 6772 | 28.2 |
| 2 | 5,102,910 | 11 | 515 | 6362 | 31.4 |

### Example 8: Identification of Suitable Frits for Microbead Bed Formation

In Examples 3, the microbead bed was prepared by applying a microbead suspension to a column that had been blocked on the opposite end by a frit which retained the microbeads within the column. The microbeads were applied under pressure which aids formation of the microbead bed. The sample as well as hybridisation and wash buffers were also applied under pressure to maintain the optimal configuration of the microbead bed and to control the flow rates through the microbead bed, making it possible to optimize hybridisation and washing conditions.

Many commercially available DNA or RNA purification columns are either designed for gravity flow or to withstand centrifugation at high-speed. Most of these columns are not suitable for use with the set-up described in Example 3 because they either do not retain the sample for a sufficient amount of time (in case of gravity flow), making it difficult to control hybridisation and washing conditions, or they require high pressure to force fluids through (e g. spin columns), resulting in unsuitably low flow rates under the low pressure conditions used in Example 3.

### Frit Requirements

To identify a frit material suitable for the microfluidic applications described in Example 3, several different materials from various suppliers were tested. Each material had to meet the following requirements to be included in the tests:
hydrophilic to aid wetting
Low DNA binding capacity
suitable for use in DNA/RNA purification.
DNA/RNA-free
appropriate pore sizes to be able to retain beads of >5 μm size (bead size used in Example 3).
able to withstand maximum temperature of 95° C.

### Tests

To prepare the test material for insertion into an empty column, a cylinder was cut out with a punch (diameter: 2.5 mm) to form a frit. The newly formed frit was inserted in an empty plastic column with tweezers. The column was then placed vertically into a rig. Two tests were performed. In the first test, 500 μl of water was pipetted into the column. The water level was marked with a felt-tip pen. After 300 seconds, the column was photographed to record the water level. The tested material was considered suitable for use as a frit if no flow occurred when no pressure was applied for a period of 300 seconds.

In the second test, a vacuum pump set at 50 mbar was connected to the base of the column. 500 μl of water was pipetted into the column and the water level was marked in the column again (if required). Another 500 μl of water was pipetted into the column and the vacuum pump was turned on and a timer was started. The timer was stopped when the water level had reached the 500 μl mark and the time was recorded. The tested material was considered suitable for use as a frit if it allowed flow rates of 5-100 μl/min at 50 mbar pressure.

### Results

A range of 18 frit materials were tested. Two frit types were identified that met the suitability requirements and were found to be particularly suitable for practising the invention. The frit characteristics are summarized in Table 8:

TABLE 8

| Reference | Make | Pore Size | Thickness | Material |
|---|---|---|---|---|
| Frit type 1 | Porex | 7-12 μm | 2.0 mm | XS-82591 |
| Frit type 2 | ROBU | 4-5.5 μm | 2.5 mm | VitraPOR |

Frit type 1 was made of a hydrophilic polyethylene sheet with small pore sizes (7-12 μm) to prevent leakage. This material is chemically compatible with 95% ethanol and 0.1N NaOH and can withstand a maximum temperature of 121° C., which makes it suitable for autoclaving. Frits prepared from this material were relatively easy to assemble into columns. Use of frit type 1 resulted in a good flow rate (30-40 μl/min) at 50 mbar pressure and good microbead bed characteristics. Despite the slightly larger pore size, it had a good retention capacity for the beads. Frit type 1 was used for the set-up described in Example 4.

Frit type 2 was made of borosilicate glass. This material can withstand temperatures of up to 515° C. and is chemically compatible with water, acids and alkalis, salt, organic substances, chlorine and bromine. This frit type showed characteristics similar to frit type 1 in terms of flow rate, microbead bed characteristics and bead retention capacity. Frits prepared from this material were solid, maintained their shape when assembled into columns and had a flat top surface after insertion in the columns.

It will be understood that the invention has been described by way of example only and modifications may be made whilst remaining within the scope and spirit of the invention.

### REFERENCES

[1] Wei et al., Nucleic Acids Res. 2005 May 12; 33(8):e78.
[2] Peytavi et al., Clin Chem. 2005 October; 51(10):1836-44.
[3] Carter et al., Nucleic Acids Res. 2007; 35(10):e74.
[4] Kim et al., Langmuir. 2006 Nov. 21; 22(24):10130-4.
[5] Summerer et al., Genome Res. 2009 September; 19(9):1616-21.
[6] Albert et al., Nat Methods. 2007 November; 4(11):903-5.
[7] Hodges et al., Nat Protoc. 2009; 4(6):960-74.
[8] US 2013/0130917
[9] Knierim et al., PLoS One. 2011; 6(11):e28240
[10] Poptsova et al., Sci Rep. 2014 Mar. 31; 4:4532
[11] Supelco Technical Bulleting 862B, Sigma-Aldrich Co. 1996
[12] Vialard et al., Prenat Diagn. 2011 May; 31(5):500-8
[13] Garcia-Herrero et al., Biomed Res Int. 2014; 2014:590298
[14] Manning et al., Genet Med. 2010 November; 12(11):742-5
[15] Wincent et al., Clin Genet. 2011 February; 79(2):147-57
[16] Mockler et al., Genomics. 2005 January; 85(1):1-15
[17] U.S. Pat. No. 6,770,751
[18] Parkinson et al., Genome Res. 2012 January; 22(1):125-33
[19] Sanger et al., Proc Natl Acad Sci USA. 1977 December; 74(12):5463-7
[20] Maxam & Gilbert, Proc Natl Acad Sci USA. 1977 February; 74(2):560-4
[21] Mardis et al., Annu Rev Anal Chem (Palo Alto Calif). 2013; 6:287-303
[22] Buermans & den Dunnen, Biochim Biophys Acta. 2014 Jul. 1. pii: 50925-4439(14)00180-X
[23] Li, arXiv:1303.3997v2 [q-bio.GN] (2013).
[24] McKenna et al., Genome Res. 2010 September; 20(9):1297-303.
[25] Li et al., Bioinformatics. 2009 Aug. 15; 25(16):2078-9
[26] Sherry et al., Nucleic Acids Res. 2001 Jan. 1; 29(1):308-11.
[27] Danecek et al., Bioinformatics. 2011 Aug. 1; 27(15):2156-8.
[28] McLaren et al., Bioinformatics. 2010 Aug. 15; 26(16):2069-70.

1-56. (canceled)

57. A hybridisation column comprising an inner channel, wherein a portion of said channel is filled with a porous solid support comprising:
(i) a plurality of interconnected, micron-sized voids that permit a fluid to flow between them and the remainder of the channel, and
(ii) a plurality of hybridisation probes, which are bound to the surfaces of the solid support forming the voids,
wherein the pore structure of the solid support is homogeneous in all dimensions, wherein the solid support fills the entire cross-section of the channel.

58. The hybridisation column of claim 57, wherein the voids have an average pore size of 0.1-100 μm.

59. The hybridisation column of claim 57, wherein the portion of the channel occupied by the solid support has a volume ranging from 0.1 mm³ to 100 mm³.

60. The hybridisation column of claim 57, wherein the voids within the solid support take up about 30-50% of the volume taken up by the solid support in the channel.

61. The hybridisation column of claim 57, wherein one end of the channel comprises a porous filter, frit or permeable membrane to keep the solid support in the column when suction is applied to said end of the channel or pressure is applied to the opposite end of the channel.

62. A microfluidic device comprising one or more hybridisation column(s) according to claim 57, a temperature control element and a temperature sensor, wherein the temperature control element can be used to control the temperature within the channel of the one or more hybridisation column(s).

63. The microfluidic device of claim 62, further comprising one or more reservoir(s) wherein each reservoir is connected to one end of the channel of the one or more hybridisation column(s), wherein each reservoir can be sealed and pressurised.

64. The microfluidic device of claim 63, wherein the pressure is supplied by a source of compressed gas, which is connected to the one or more reservoir(s) via tubing.

65. The microfluidic device of claim 64, wherein pressure applied to the one or more sealed reservoir(s) pushes a fluid within the channel of the one or more hybridisation columns through the solid support.

66. The microfluidic device of claim 64, further comprising a valve within the tubing that connects the source of compressed gas supply to the one or more reservoir(s), wherein the valve can be opened and closed by a controller, which can control the flow rate of fluid driven through the channel of the one or more hybridisation column(s).

67. The microfluidic device of claim 63, wherein the pressure is supplied by a pump, wherein the pump is connected to an electronic controller, which can be programmed to control the flow rate of fluid driven through the channel of the one or more hybridisation column(s).

68. The microfluidic device of claim 63, further comprising one or more collection tubing attached to the other end of the channel of the one or more hybridisation columns.

69. The microfluidic device of claim 64, wherein one end of the channel of the one or more hybridisation columns is connected to a suction pump, wherein the suction pump is connected to a controller, which can control the flow rate of fluid driven through the channel of the one or more hybridisation column(s) when suction is applied.

70. A microfluidic device adapted for receiving the column of claim 57 in order to produce a device comprising one or more hybridisation column(s), a temperature control element and a temperature sensor, wherein the temperature control element can be used to control the temperature within the channel of the one or more hybridisation column(s).

71. A method of preparing the microfluidic device of claim 62 by inserting a hybridisation column comprising an inner channel, wherein a portion of said channel is filled with a porous solid support comprising:
(i) a plurality of interconnected, micron-sized voids that permit a fluid to flow between them and the remainder of the channel, and
(ii) a plurality of hybridisation probes, which are bound to the surfaces of the solid support forming the voids,
wherein the pore structure of the solid support is homogeneous in all dimensions, wherein the solid support fills the entire cross-section of the channel.

72. A method for enriching nucleic acid molecules from a complex mixture of nucleic acids, wherein said method comprises:
(i) providing a porous solid support comprising
(a) a plurality of interconnected, micron-sized voids that permit a fluid to flow between them, and
(b) a plurality of hybridisation probes, which are bound to the surfaces of the solid support forming the voids,
(ii) driving the mixture of nucleic acids through the solid support thereby allowing nucleic acid molecules comprising nucleic acid sequences complementary to the nucleic acid sequences of the hybridisation probes to hybridise to the probes, wherein hybridisation is allowed to proceed for less than 10 hours,
(iii) washing the solid support to remove any nucleic acids that are not hybridised to the probes, and
(iv) eluting the nucleic acid molecules bound to the hybridisation probes from the solid support.

73. The method of claim 72, wherein the individual hybridisation probes for each complementary nucleic acid sequence are randomly distributed on the surface of the solid support.

74. The method of claim 72, wherein the mixture is driven through the solid support at a flow rate of 1-100 μl/minute.

75. The method of claim 72, wherein hybridisation takes place at a temperature of 55-65° C.

76. The method of claim 75, wherein the washing step is performed at a temperature 5-10° C. below the temperature used for hybridisation.

77. The method of claim 72, wherein elution is achieved by heating the solid support to a temperature of about 90-100° C.

78. The method of claim 72, wherein the hybridisation probes are 50-250-mer oligonucleotide of the same length and/or the nucleic acid molecules in the mixture have a length of 100-350.

79. The method of 72, wherein the mixture is prepared by obtaining a larger nucleic acid molecule, fragmenting it into nucleic acid fragments, and joining the fragments to adaptor molecules.

80. A method of preparing the hybridisation column of claim 57, wherein the method comprises:
(i) providing a column comprising an inner channel, and
(ii) filling the entire cross-section of the channel with a plurality of microbeads having about the same diameter and having linked to their surfaces a plurality of hybridisation probes, wherein the microbeads form a porous solid support.

81. The method of claim 80, wherein the diameter is 0.5 to 500 μm.

82. A kit for preparing the hybridisation columns of claim 57, wherein the kit comprises (i) one or more column(s) comprising a inner channel and (i) a container comprising a plurality of microbeads having about the same diameter and having linked to their surfaces a plurality of hybridisation probes.


SEQUENCE LISTING (11 sequences)

SEQ ID NO: 1 - DNA, 60 bp, Artificial Sequence
  Oligonucleotide
tgaggcttgcataatggcattcagaatgagtgaacaaccacggaccataaaaatttataa

SEQ ID NO: 2 - DNA, 100 bp, Artificial Sequence
  Oligonucleotide
cattagttccggccagcagattataaatttttatggtccgtggttgttcactcattctgaatgccattatgcaagcctcacaatatagttaaatgcaatg

SEQ ID NO: 3 - DNA, 100 bp, Artificial Sequence
  Oligonucleotide
gagttgcccatcgatatgggcaactctatctgcactgctcattaatatacttctgggttccttccagttgtttttgcatagtgatcagcctctctctgag

SEQ ID NO: 4 - DNA, 120 bp, Artificial Sequence
  Oligonucleotide
ccgtcaaaaacattgcatttaactatattgtgaggcttgcataatggcattcagaatgagtgaacaaccacggaccataaaaatttataatctgctggccggaactaatgaatttattgg

SEQ ID NO: 5 - DNA, 31 bp, Artificial Sequence
  Oligonucleotide
actctttccctacacgacgctcttccgatct

SEQ ID NO: 6 - DNA, 30 bp, Artificial Sequence
  Oligonucleotide
gatcggaagagcgtcgtgtagggaaagagt

SEQ ID NO: 7 - DNA, 28 bp, Artificial Sequence
  Oligonucleotide
gatcggaagagcacacgtctgaactcca

SEQ ID NO: 8 - DNA, 29 bp, Artificial Sequence
  Oligonucleotide
tggagttcagacgtgtgctcttccgatct

SEQ ID NO: 9 - DNA, 57 bp, Artificial Sequence
  Oligonucleotide
aatgatacggcgaccaccgagatctacactctttccctacacgacgctcttccgatc

SEQ ID NO: 10 - DNA, 55 bp, Artificial Sequence
  Oligonucleotide
caagcagaagacggcatacgagatgcacttgtgactggagttcagacgtgtgctc

SEQ ID NO: 11 - DNA, 55 bp, Artificial Sequence
  Oligonucleotide
caagcagaagacggcatacgagatcgtgatgtgactggagttcagacgtgtgctc